(19) United States
(12) Patent Application Publication
(10) Pub. No.: US 2006/0251750 A1
(43) Pub. Date: Nov. 9, 2006

(54) Soy formulations and their use in skin care

(76) Inventor: Aaron Thomas Tabor, Winston-Salem, NC (US)

Correspondence Address: KILPATRICK STOCKTON LLP, 1001 WEST FOURTH STREET, WINSTON-SALEM, NC, 27101, US

(21) Appl. No.: 11/480,238

(22) Filed: Jun. 30, 2006

Related U.S. Application Data
(63) Continuation-in-part of application No. 10/260,993, filed on Sep. 30, 2002.

(51) Int. Cl. A61K 36/48; A61K 31/4188

(52) U.S. Cl. 424/757; 514/393

(57) ABSTRACT

Disclosed are methods for maintaining and/or improving the quality of at least one of epidermal, dermal or subcutaneous tissue in a subject. The subject may, in certain embodiments, be a post-menopausal human. The methods may comprise application of a soy formulation to the subject. Also disclosed are soy formulations, dietary supplements, food products, and pharmacological compositions that may be useful for maintaining and/or improving the quality of at least one of epidermal, dermal or subcutaneous tissue, including skin, hair, and nails.

### STATEMENT OF RELATED APPLICATIONS

The present application claims priority from U.S. provisional patent application Ser. No. 60/092,985, filed Jul. 16, 1998, U.S. provisional patent application Ser. No. 60/105,797, filed Oct. 27, 1998, and U.S. nonprovisional patent application Ser. No. 09/356,167, filed Jul. 16, 1999, now U.S. Pat. No. 6,482,448, U.S. nonprovisional patent application Ser. No. 10/260,993, filed Sep. 30, 2002. The disclosure of each of these applications is hereby incorporated herein by reference.

### FIELD OF THE INVENTION

The present invention relates to soy formulations and the use of such formulations in skin care.

### BACKGROUND

Soybeans include natural plant estrogens, known as phytoestrogens or isoflavones. These isoflavones are thought to bind to estrogen receptors and thus exert an estrogenic response. Medical studies have shown that isoflavones in soy protein may have many beneficial interactions with a variety of human tissues, are safe, cause no significant side-effects, and are the primary reason for many health benefits. In Asian countries like Japan, where the traditional diet is high in soy protein, it has been found that very few women complain about menopausal symptoms; for example only 9% of Japanese women complain about hot flashes. In addition, younger women have fewer menstrual periods per year, and occurrences of some cancers (breast and endometrial), heart disease, and other chronic diseases are very low. It has been reported that Japanese women consume up to 200 mg of isoflavones in their daily diet and have a lower incidence of perimenstrual and menopausal symptoms than women in other cultures consuming less daily dietary isoflavones. Previous studies using isoflavone dosages of 76 mg per day report a 45% reduction in hot flashes in women after 12 weeks.

In addition to providing isoflavones, soybeans provide a source of protein. It is believed that the benefits from soy come from the phytoestrogens (isoflavones), other compounds such as lignans and saponins, the soy protein itself, and undoubtedly undiscovered compounds.

Also, recent research has suggested that soy and/or soy isoflavones may be beneficial for a variety of dermatologic problems. For example, dietary consumption of soy oil reduced the incidence of alopecia areata (hair loss) in a dose-response fashion in a mouse model (McElwee et al., Exp. Dermatol., 2003, 12:30-36). The effect of soy on hair loss may be caused at least in part by non-isoflavone components of the soy, as an immunostimulating peptide, “soymetide-4” isolated from a trypsin digest of soy protein was found to inhibit chemotherapy-induced alopecia in neonatal rats (Tsuruki et al., J. Invest. Dermatol., 2004, 122:848-850).

Also, soy may have protective effects against photoaging and photocarcinogenesis. Administration of soy extract may reduce transepidermal water loss, fine wrinkles, and UV radiation-induced thickening of the epidermis (Kim et al., J. Amer. Coll. Nutr., 2004, 23:15-162). Other studies have shown similar beneficial effects of isoflavones. For example, lotions of genistein, equol, isoequol and dehydroequol may reduce inflammatory edema reactions and the suppression of contact hypersensitivity induced by UV radiation (Widyarini et al., Photochem. Photobiol., 2001, 74:465-470). It has also been shown that daily topical applications of equol lotions may reduce the proportion of tumors progressing from benign papillomas to malignant squamous cell carcinomas, and may reduce the size of these carcinomas (Widyarini et al., Photochem. Photobiol., 2004). Also, in hairless mice, both topical and oral administration of genistein may inhibit the initiation and promotion of photocarcinogenesis, resulting in the reduction of tumor incidence and multiplicity (Wei et al., J. Nutr., 2003, 133:3811S-3819S).

Several soy protein formulations are commercially available. Formulations produced exclusively from the whole soy bean generally include 1-5 milligrams isoflavone per gram of the formulation and may be up to 90% protein. Formulations produced exclusively from the heart (center) of the soy bean generally include 24-36 milligram isoflavone per gram of the formulation. Although tablets with concentrations of 100-750 milligram isoflavones per gram are available, formulations having concentrations greater than 36 mg isoflavones per gram of the formulation are chemically extracted and modified and therefore are not considered natural.

Dietary levels of 60 milligrams, in particular 120-200 milligrams of isoflavones can be difficult to achieve utilizing currently available natural soy protein formulations. To achieve a dietary level of greater than 60 milligrams, and in particular 120-200 milligrams of isoflavones from soy formulations produced utilizing the whole soy bean could require ingesting greater than 60 grams, in particular up to 120-200 grams of soy protein. Intake of soy protein at this volume level may cause digestive side effects such as bloating and constipation. To achieve a dietary level of greater than 60 milligrams, or about 60-200, or 120-200 milligrams, of isoflavones from soy formulations produced utilizing the heart of the soy bean may require ingesting only 3 to 8 grams of soy protein. However, with soy protein levels this low, the person ingesting the formulation would be receiving minimal benefits and nutritional value from soy protein itself.

Accordingly, it would be advantageous to have a natural soy formulation having a milligram isoflavone to gram of the formulation ratio falling between the ratios of natural soy formulations produced from the whole soybean and natural soy formulations produced from the heart of the soy bean.

It would also be advantageous to have dietary supplements that include the natural soy formulations. In particular, it may be advantageous to have dietary supplements which when ingested in reasonable amounts provide dietary isoflavone levels of greater than 60 milligrams, or greater than 120 milligrams, for example 120-200 milligrams or higher, e.g. greater than 200 milligrams. For certain applications it may be advantageous to have dietary supplements or soy formulations that provide dietary isoflavone levels as high as 1500-2200 milligrams, or greater.

Further, it would be advantageous to have food products that include natural soy formulations and/or natural dietary supplements. In particular, it may also be advantageous to have food products which when ingested in reasonable amounts provide dietary isoflavone levels of greater than 60 milligrams, or greater than 120 milligrams, for example 120-200 milligrams, or higher, e.g., greater than 200 milligrams, up to 400-900 milligrams or greater. For certain applications it may be advantageous to have food products, dietary supplements or soy formulations that provide dietary isoflavone levels as high as 1500 to 2200 milligrams, or greater.

In addition to the known benefits of soy described above, it may be advantageous to have a formulation that may be used to maintain and/or improve the appearance of skin, hair and/or nails. Thus, in many instances, there is a need for a soy formulation that will improve the appearance of skin, hair and/or nails. It may also be advantageous to have a soy formulation that may improve the appearance of skin, hair, and/or nails in populations that require other beneficial effects of soy phytoestrogens and/or soy protein.

These and other advantages are achieved by the present invention.

### SUMMARY OF THE INVENTION

Embodiments of the present invention may comprise a soy formulation that is useful for maintaining and/or improving the quality of at least one of epidermal, dermal or subcutaneous tissue, including skin, hair, and nails. In certain embodiments, the present invention may comprise soy protein formulations (soy formulations), dietary supplements, and/or food products, where the dietary supplements and/or food products comprise the soy formulations of the present invention. In other embodiments, the present invention may comprise pharmacological compositions that is useful for maintaining and/or improving the quality of at least one of epidermal, dermal or subcutaneous tissue, including skin, hair, and nails.

The present invention also comprises methods for using such formulations, dietary supplements, food products, and pharmacological compositions to maintain and/or improve the quality of at least one of epidermal, dermal, or subcutaneous tissue in a subject. In an embodiment, the epidermal, dermal or subcutaneous tissue may comprise skin, hair, and nails.

Thus, in certain embodiments, the present invention comprises a soy formulation that is useful for maintaining and/or improving the quality of at least one of epidermal, dermal or subcutaneous tissue, and methods of using such formulations. In an embodiment, the soy formulation is natural. Embodiments of the soy formulations of the present invention may include a higher concentration of natural plant estrogens, referred to as isoflavones or phytoestrogens, per gram of the formulation, than were available in a natural product prior to the discovery of the soy formulations described herein. Embodiments of the soy formulations of the present invention may include protein and other healthful components derived from the soybean, thereby providing a useful mixture of isoflavones and protein, with higher levels of isoflavones or phytoestrogens than were available in a natural product prior to the discovery of the soy formulations described herein.

In other embodiments, the present invention may comprise a dietary supplement comprising a soy formulation that is useful for maintaining and/or improving the quality of at least one of epidermal, dermal or subcutaneous tissue, and methods of using such dietary supplements. The dietary supplement may further comprise ingredients such as enzymes, a fiber source, vitamins and the like. In an embodiment, the soy formulation may be natural.

In yet other embodiments, the present invention may comprise a food product comprising a soy formulation and/or a dietary supplement of the present invention wherein the soy formulation and/or dietary supplement is useful for maintaining and/or improving the quality of at least one of epidermal, dermal or subcutaneous tissue, and methods of using such food products. In an embodiment, the soy formulation may be natural. The food product may further comprise additional ingredients.

Embodiments of the present invention may also comprise pharmacological compositions. For example, a pharmacological composition of the present invention may comprise a soy formulation of the present invention in a pharmacologically effective amount that is useful for maintaining and/or improving the quality of at least one of epidermal, dermal or subcutaneous tissue, including skin, hair, and nails, and methods of using such pharmacological compositions. In an embodiment, the soy formulation may be natural. The compositions may, in certain embodiments, comprise additional pharmacologically active compounds. In some embodiments, the additional pharmacologically active compound may comprise a medicinal agent, such as a prescription medicine.

In other embodiments, the present invention may comprise methods of maintaining and/or improving the quality of at least one of epidermal, dermal or subcutaneous tissue such as skin, hair, or nails, in a subject in need thereof. The method may comprise application of a soy formulation, dietary supplement, food product, or pharmacological composition of the present invention. In an embodiment, the soy formulation may be natural. In one embodiment, the soy formulation may comprising a first portion produced from the heart of a soybean, and a second portion produced from a whole soy bean, wherein the soy formulation is effective to maintain and/or improve the quality of at least one of epidermal, dermal, or subcutaneous tissue. In an embodiment, the soy formulation comprises an amount of isoflavone, protein and/or other components of the soybean that are effective to maintain and/or improve the quality of at least one of epidermal, dermal, or subcutaneous tissue.

A variety of subjects may be targeted by the methods of the present invention. In one embodiment, the subject may be a human.

Embodiments of the present invention may provide certain advantages.

For example, embodiments of the soy formulations, dietary supplements, food products and/or pharmacological compositions of the present invention may advantageously be utilized in methods for promoting healthy skin, hair or nails in an individual.

Also, embodiments of the present invention may provide advantages in that the soy formulations, dietary supplements, food products, and pharmacological compositions of the present invention may provide higher concentrations of isoflavones in a readily digestible and absorbable form than soy products available prior to the discovery of the compositions described herein.

Also the pharmacological compositions of the present invention may, in certain embodiments, provide synergistic effects. Thus, in certain embodiments, the combination of the soy formulation and a medicinal agent may advantageously produce one or more of the following effects:
1) additive and/or synergistic benefits;
2) reduction of the side effects and/or adverse effects associated with use of the additional pharmacologically active compound in the absence of the soy formulation; and/or
3) the ability to lower the dosage of the pharmacologically active compound in comparison to the amount of prescription medicine needed in the absence of the soy formulation.

As another potential advantage, the soy formulations, dietary supplements, food products and pharmacological compositions of the present invention may also provide protein. The isoflavones and/or protein provided by the soy formulations, dietary supplements, food products and pharmacological compositions of the present invention may provide numerous benefits to the skin, hair and nails of an individual. In particular, the soy formulations, dietary supplements, food products and pharmacological compositions of the present invention may be ingested in amounts that provide isoflavones in amounts that are greater than 60 milligrams, and in certain embodiments, greater than 200 milligrams, or greater than 1800 milligrams, and that may additionally provide greater than 20 grams of protein, without the unpleasant side effects individuals have experienced with prior products.

Further advantages and possible embodiments of the present invention are discussed herein.

### BRIEF DESCRIPTION OF THE FIGURES

FIG. 1 shows the effects on skin after consumption of a soy formulation of the present invention after 6 months (top bar of each pair) and 3 months (bottom bar of each pair) in accordance with alternate embodiments of the present invention. Actual percentages are listed by each bar.

FIG. 2 shows the effects on hair after consumption of a soy formulation of the present invention after 6 months (top bar of each pair) and 3 months (bottom bar of each pair) in accordance with alternate embodiments of the present invention. Actual percentages are listed by each bar.

FIG. 3 shows the effects on nails after consumption of a soy formulation after 6 months (top bar of each pair) and 3 months (bottom bar of each pair) in accordance with alternate embodiments of the present invention. Actual percentages are listed by each bar.

### DETAILED DESCRIPTION OF THE INVENTION

Definitions

As used herein, epidermal tissue comprises tissue derived from the ectoderm. The ectoderm is the outermost germ layer of metazoan embryos, developing into epidermal and nervous tissue. Epidermal tissue includes skin, nails, and hair.

As used herein, skin includes the epidermis and the dermis. The skin may also be considered to include the subcutaneous layer. The outermost epidermis in skin consists of stratified squamous epithelium with an underlying basement membrane. The epidermis in skin does not contain any blood vessels, but receives nutrients by diffusion from the dermis. The main types of cells that make up the epidermis are keratinocytes. Also, melanocytes and Langerhans cells are also present. The epidermis can be further subdivided into the following layers from outermost to innermost: corneum, lucidum, granulosum, spinosum, and basale.

The dermis lies below the epidermis and includes blood vessels, nerves, hair follicles, smooth mucscle, glands and lymphatic tissue. The dermis includes a sensitive connective tissue layer of the skin located below the epidermis, containing nerve endings, sweat and sebaceous glands, and blood and lymph vessels. The dermis is also called corium, cutis, or derma.

The skin, as used herein, may also include the subcutaneous tissue. The subcutaneous tissue layer contains fat and connective tissue that houses larger blood vessels and nerves. The subcutaneous layer may be important is the regulation of temperature of the skin itself and the body. The size of the subcutaneous layer varies throughout the body and from person to person.

Also, as used herein, a subject in need of maintaining and/or improving the quality of at least one of his or her epidermal, dermal, or subcutaneous tissue is a mammal who may require treatment to maintain and/or to improve the condition of his or her skin, hair or nails. Such subjects may include animals (e.g., animals who may have a skin condition) and humans. Conditions that may be treated by the products and/or methods of the invention may include those that are inherent to the subject's skin, hair or nails (e.g., baldness), problems that are secondary to other treatments (e.g., drug-induced acne), and/or conditions brought on by aging or exposure of the epidermal tissue to environmental elements (e.g., sun and or dry air). In an embodiment, the subject may comprise a human. In an embodiment, the subject may comprise a post-menopausal female. Or the subject may comprise a non-human mammal as for example, pets that may have a need for treatment of the skin, nails, hair, and/or fur.

As used herein, “natural” comprises ingredients that are derived from nature and are not chemically extracted and/or modified.

The term “treating” or “treat” refers to improving a symptom of a disease or disorder and may comprise curing the disorder, substantially preventing the onset of the disorder, or improving the subject's condition. The term “treatment” as used herein, refers to the full spectrum of treatments for a given disorder from which the patient is suffering, including alleviation of one symptom or most of the symptoms resulting from that disorder, a cure for the particular disorder, or prevention of the onset of the disorder.

As used herein, the term “maintaining and/or improving the quality of at least one of epidermal, dermal or subcutaneous tissue” means to perform an action with the ensuing result that the tissue, by qualitative or quantitative assessment, has the desired characteristics associated with healthy skin, hair or nails. Aspects which contribute to the health of epidermal, dermal, or subcutaneous tissue include the following: a reduction or lack of wrinkling of the skin; a reduction or lack of a decrease in skin thickness; a reduction or lack of areas of undesired discoloration of the skin; a reduction or lack of irritation, redness, or inflammation of the skin; a reduction or lack of undesired dryness or oiliness of the skin; a reduction or lack of flaking in the skin; a reduction or lack of roughness of the skin; a reduction or lack of undesired tightness and firmness of the skin; a reduction or lack of acne; a reduction or lack of sagging of the skin; a reduction or lack of splitting, ridging, flaking, breaking, roughness, and slow growth in the nails; or a reduction or lack of splitting roughness, dullness, thinning, slow growth of the hair; and a desired thickness and manageability in the hair. Monitoring of these qualities may be performed using the methods described herein, or other methods known in the art.

As used herein, a desired therapeutic effect is a therapeutic result that benefits the subject. Specific therapeutic effects may include maintaining and improving the quality of at least one of epidermal, dermal or subcutaneous tissue (e.g., skin, hair, or nails) in a subject. Also, specific therapeutic effects may include treatment of symptoms associated with menopause (including and or pre-menstrual syndrome (PMS)) as described herein). Specific therapeutic effects may also include relieving symptoms associated with cardiovascular disorders, bone and skeletal disorders (i.e., including osteoporosis and disorders of joints and cartilage), breast and prostate-related conditions, endometrial-related disorders, disorders related to the nervous system and/or brain, baldness, gastrointestinal conditions, skin conditions, kidney-related conditions, lung and breathing conditions, immune conditions, reproductive conditions, diabetes, eye conditions, obesity, sexual dysfunction, chronic fatigue, hypoglycemia, or hyperglycemia.

Notwithstanding that the numerical ranges and parameters setting forth the broad scope of the invention are approximations, the numerical values set forth in the specific examples are reported as precisely as possible. Any numerical value, however, may inherently contain certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

Soy Formulations

Embodiments of the present invention may comprise a soy formulation that is useful for maintaining and/or improving the quality of at least one of epidermal, dermal or subcutaneous tissue, including skin, hair, and nails in a subject in need thereof. The formulation may comprise an amount of isoflavone, protein, and/or other components of the soybean in variety of ranges that may be useful for maintaining and/or improving the quality of at least one of epidermal, dermal or subcutaneous tissue. Thus, in one embodiment, the present invention may comprise a composition comprising a soy formulation comprising: a first portion produced from the heart of a soybean; and a second portion produced from a whole soy bean; wherein the soy formulation comprises an amount of isoflavone that is effective to maintain and/or improve the quality of at least one of epidermal, dermal or subcutaneous tissue. The formulation may be applied to the subject topically (for example, as a cream, gel or lotion), as part of a medicament, or may be ingested as a dietary supplement or other food product as described herein.

In certain embodiments, the soy formulations of the present invention may include a defined range of natural plant estrogens (referred to herein as isoflavones or phytoestrogens) per gram of protein. In an embodiment, the amount of isoflavones and soy protein may be varied by altering the relative amounts of the soybean heart and rest of the soybean used to prepare the formulation. For example, in alternate embodiments, a soy formulation of the present invention may comprise about 3 to 23 milligrams of at least one isoflavone per gram of the formulation, or about 5 to 15 milligrams of at least one isoflavone per gram of the formulation, or about 6 to 9 milligrams of at least one isoflavone per gram of the formulation.

Embodiments of the isoflavone component of the soy formulation of the present invention may comprise various known and naturally occurring isoflavones. In various embodiments, the naturally occurring isoflavones may comprise at least one of diadzin, genistin, or glycitin. For example, embodiments of the present invention comprise a soy formulation comprising diadzin, genistin and glycitin and having a diadzin to genistin to glycitin ratio of between about 3:1:2 and 3:4.5:1. Thus, in these embodiments, the soy formulation may comprise about 3 parts diadzin; 1 to 4.5 parts genistin; and 1 to 2 parts glycitin. In certain embodiments, a soy formulation of the present invention may have a ratio of diadzin to genistin to glycitin of approximately 2:1:1, respectively, such that diadzin is the major isoflavone component.

Although the isoflavones discussed herein are discussed with reference to their glycone forms, the present invention may utilize aglycone forms of isoflavones which may be digested and/or absorbed more easily by an individual. As is known in the art, the aglycone form refers to glycone after cleavage of the glucose subgroup.

The soy formulations of the present invention may comprise various amounts of soy protein. For example, in alternate embodiments, the soy formulations of the present invention may comprise about 0.4 to 1.2 grams, or about 0.4 to 0.9 grams, or abut 0.6 to 0.8 grams of protein per gram of the formulation. Thus, in alternate embodiments, a soy formulation of the present invention may comprise: (i) about 3 to 23 milligrams, or 5 to 15 milligrams, or 6 to 9 milligrams of at least one isoflavone per gram of the formulation; and (ii) about 0.4 to 1.2 grams, or 0.4 to 0.9 grams, or 0.6 to 0.8 grams protein per gram of the formulation.

In one embodiment, the present invention may comprise a soy formulation produced by combining a first portion of higher isoflavone concentration soy product produced from the heart of the soy bean and a second portion of lower isoflavone concentration soy product produced from the whole soy bean. When prepared in this manner, the formulation may comprise, in alternate embodiments, about 3 to 23 milligrams of at least one isoflavone per gram of the formulation, or about 5 to 15 milligrams of at least one isoflavone per gram of the formulation, or about 6 to 9 milligrams of at least one isoflavone per gram of the formulation. The isoflavone(s) may, in certain embodiments, comprise diadzin, genistin and glycitin. When prepared in this manner, the soy formulation may comprise, in alternate embodiments, about 0.4 to about 1.2 grams, or about 0.4 to 0.9 grams, or about 0.6 to 0.8 grams protein per gram of the formulation. Also, in certain embodiments, the soy formulation may include the diadzin-genistin-glycitin ratios as described herein.

Also, the formulation may include additional compounds. In an embodiment, biotin may be included. For example, the soy formulation may comprise an amount of biotin such that one dose (or serving) of the formulation comprises about 0.5 to 20, or 1 to 10, or about 2.5 mg of biotin.

In an embodiment, the soy formulation used for skin treatment may comprise a multi-vitamin or a vitamin supplement. In alternate embodiments, the soy formulation or multi-vitamin used in the soy formulation may comprise at least one of a multi-vitamin/multi-mineral supplement, an antioxidant supplement, an amino acid supplement, a skin matrix support supplement, a skin clearing supplement, a fatty acid supplement, or a mood-enhancing supplement. For example, a multi-vitamin or supplements as described herein (Table 1) may be included.

A soy formulation of the present invention may take many forms. In one embodiment, the soy formulations of the present invention may be in powder form. Alternatively, the soy formulations may be in tablet or liquid form. Or, the soy formulation of the present invention may comprise a cream, gel or lotion for topical application. Also, and as discussed in detail herein, the soy formulations of the present invention may be included within a dietary supplement, or within food items, such as nutrition bars, liquid drinks, cereals etc., in a food product of the present invention.

Dietary supplements

The present invention may also comprise soy formulations utilized in dietary supplements. The dietary supplements may comprise an amount of the soy formulation so as to be useful for maintaining and/or improving the quality of at least one of epidermal, dermal or subcutaneous tissue, including skin, hair, and nails. In certain embodiments, the dietary supplements of the present invention may include a defined range of natural plant estrogens (referred to herein as isoflavones or phytoestrogens) per gram of protein. The dietary supplement may comprise an amount of isoflavone, protein, and/or other components of the soybean in variety of ranges that may be useful for maintaining and/or improving the quality of at least one of epidermal, dermal or subcutaneous tissue. In an embodiment, the amount of isoflavones and soy protein may be varied by altering the relative amounts of the soybean heart and the rest of the soybean used to prepare the supplement.

The dietary supplement may comprise a soy formulation having one or more of the features described above. The amount of soy formulation utilized in a dietary supplement of the present invention may depend on the level of isoflavones desired per serving or dose of the dietary supplement.

For example, it may be desirable for the dietary supplement to provide greater than about 60 milligrams of at least one isoflavone per serving or dose. For example, in an embodiment, a serving of a dietary supplement of the present invention may comprise 10 grams of a soy formulation of the present invention. The soy formulation may comprise about 7 milligrams of at least one isoflavone per gram of the formulation, so as to provide about 70 milligrams of isoflavones in the 10 gram serving of the dietary supplement. To achieve higher levels of isoflavones, the dietary supplements of the present invention may include additional amounts of a soy formulation having a higher level of isoflavones per gram. Thus, alternate embodiments of a dietary supplement of the present invention may comprise: a soy formulation comprising about 3 to 23 milligrams, or about 5 to 15 milligrams, or about 6 to 9 milligrams of at least one isoflavone per gram of the formulation, where the amount of the soy formulation in the dietary supplement is sufficient to provide greater than about 60 milligrams of at least one isoflavone per serving.

The dietary supplements of the present invention may comprise various amounts of soy protein. For example, in alternate embodiments, a dietary supplement of the present invention may comprise: (i) a soy formulation comprising about 3 to 23 milligrams, or about 5 to 15 milligrams, or about 6 to 9 milligrams of at least one isoflavone per gram of the formulation; and (ii) about 0.4 to 1.2 grams, or about 0.4 to 0.9 grams, or about 0.6 to 0.8 grams protein per gram of the formulation, where the amount of the soy formulation is sufficient to provide greater than about 60 milligrams of at least one isoflavone per serving.

In another embodiment, a dietary supplement of the present invention may comprise a soy formulation produced by combining a first portion of higher isoflavone concentration soy product produced from the heart of the soy bean and a second portion of lower isoflavone concentration soy product produced from the whole soy bean. In an embodiment, a supplement made in this manner may thus achieve a soy formulation comprising about 3 to 23 milligrams of at least one isoflavone per gram of the formulation, or about 5 to 15 milligrams of at least one isoflavone per gram of the formulation, or about 6 to 9 milligrams of at least one isoflavone per gram of the formulation, where the amount of the soy formulation in the dietary supplement is sufficient to provide greater than about 60 milligrams of at least one isoflavone per serving.

In certain embodiments, the dietary supplements utilize soy formulations comprising the isoflavone components and/or their ratios as described herein. In certain embodiments, the amount of soy formulation utilized in the dietary supplement of the present invention may be an amount sufficient to provide well above 60 milligrams of at least one isoflavone per serving. For example, alternate embodiments of a dietary supplement of the present invention may comprise an amount of soy formulation sufficient to provide about 120-200 milligrams of at least one isoflavone per serving, or greater than about 200 milligrams of at least one isoflavone per serving, or greater than about 400 to 900 milligrams of at least one isoflavone per serving, or greater than about 1800 milligrams of at least one isoflavone per serving, or greater than about 1800 to 2200 milligrams of at least one isoflavone per serving.

Embodiments of the dietary supplements of the present invention may comprise a single isoflavone or a plurality of isoflavones. Thus, embodiments of the dietary supplements of the present invention may include at least one of diadzin, genistin and glycitin. In an embodiment, a plurality of isoflavones including diadzin, genistin and glycitin may be used. In some embodiments, the dietary supplement may use a soy formulation that has a diadzin to genistin to glycitin ratio of between about 3:1:2 and 3:4.5:1. For example, the soy formulation may have a ratio of diadzin to genistin to glycitin of approximately 2:1:1, respectively, such that diadzin is the major isoflavone component. The dietary supplement may provide any milligram level of at least one isoflavone per serving, thus differing amounts of the soy formulation may be utilized in the dietary supplement.

There may be various combinations of soy beans, or parts of soy beans, used to make the dietary supplements of the present invention. For example, in alternate embodiments, a dietary supplement of the present invention may comprise a soy formulation comprising about 40 to 90%, or about 70 to 90% by weight protein; about 1 to 10%, or about 1 to 5% by weight fat; and about 1 to 59%, or 1 to 25% by weight carbohydrate. The analysis of protein, carbohydrate and fat levels may be accomplished utilizing conventional techniques.

A dietary supplement of the present invention may further comprise one or more additional ingredients. Thus, in alternate embodiments, a dietary supplement of the present invention may comprise at least one of a digestive enzyme, a type of fiber, a sweetener, a preservative, a mineral or the like, including calcium phosphate, soy lecithin, salt, potassium, chloride, artificial and/or natural flavorings, carragenenen, carboxymethylcellulose, xantham gum, or milk solids. Suitable digestive enzymes that may be used in the dietary supplements of the present invention include α-galactosidase. Suitable fiber sources may include, but are not limited to: psyllium. Suitable sweeteners may include, but are not limited to, natural sweeteners, including sucrose, dextrose, fructose and the like; artificial sweeteners including sucralose (SPLENDA™), aspartame, saccharin and SINETK™ (acesulfurame K) and the like; and plant derived sweeteners including stevia. The amounts of the one or more additional ingredients are such that the dietary supplement maintains the protein, carbohydrate and fat ratios set forth above.

A dietary supplement of the present invention may be in any digestible form, including a powder, a tablet or in liquid form. A dietary supplement of the present invention may also be agglomerated and/or otherwise treated to improve solubility, digestibility or other aspects of the dietary supplement.

A dietary supplement of the present invention may further include vitamins and minerals in an amount of up to 100% or more of the recommended daily allowance for each vitamin. In an embodiment, the dietary supplement may comprise biotin. For example, the dietary supplement may comprise an amount of biotin such that one dose (or serving) of the dietary supplement comprises about 0.5 to 20, or 1 to 10, or about 2.5 mg of biotin.

Alternatively or additionally, the dietary supplement may comprise a specific vitamin formulation. In an embodiment, the supplement used for skin treatment may comprise a multi-vitamin or a vitamin supplement. In alternate embodiments, the supplement or the multi-vitamin used in the supplement may comprise at least one of a multi-vitamin/multi-mineral supplement, an antioxidant supplement, an amino acid supplement, a skin matrix support supplement, a skin clearing supplement, a fatty acid supplement, or a mood-enhancing supplement. For example, a vitamin or supplements as described herein (Table 1) may be included. In one embodiment, a dietary supplement of the present invention may include 20-40% of the recommended daily allowance of most minerals.

As will be understood by those of ordinary skill in the art, a dietary supplement of the present invention may also include ingredients similar to those set forth herein with respect to a food product of the present invention.

Food Products

The present invention may also comprise soy formulations utilized in food products. The food products of the present invention may comprise an amount of the soy formulation so as to be useful for maintaining and/or improving the quality of at least one of epidermal, dermal or subcutaneous tissue, including skin, hair, and nails. In certain embodiments, the food products of the present invention may include a defined range of natural plant estrogens (referred to herein as isoflavones or phytoestrogens) per gram of protein. The food product may comprise an amount of isoflavone, protein, and/or other components of the soybean in variety of ranges that may be useful for maintaining and/or improving the quality of at least one of epidermal, dermal or subcutaneous tissue. In an embodiment, the amount of isoflavones and soy protein may be varied by altering the relative amounts of the soybean heart and the rest of the soybean used to prepare the food product.

In one embodiment, a digestible food product of the present invention may include greater than 60 milligrams of at least one isoflavone per serving. Thus, in alternate embodiments, the amount of soy formulation utilized may be an amount sufficient to provide about 120-200 milligrams of at least one isoflavone per serving; or greater than about 200 milligrams of at least one isoflavone per serving; or about 400 to 900 milligrams of at least one isoflavone per serving; or greater than about 1800 milligrams of at least one isoflavone per serving; or about 1800 to 2200 milligrams of at least one isoflavone per serving.

A food product of the present invention may comprise a soy formulation having one or more of the features described above. The amount of soy formulation utilized in a food product of the present invention may depend on the level of isoflavones desired per serving of the food product. In an embodiment, it may be desirable for the food product to provide greater than 60 milligrams of at least one isoflavone per serving. For example, a serving of a food product of the present invention may comprise 8 grams of a soy formulation of the present invention comprising 9 milligrams of at least one isoflavone per gram of the formulation, to provide 72 milligrams of isoflavones. To achieve higher levels of isoflavones, the food product may include additional amounts of a soy formulation and/or a soy formulation having a higher level of isoflavones per gram.

Thus, in alternate embodiments, a food product of the present invention may comprise a soy formulation comprising about 3 to 23 milligrams, or about 5 to 15 milligrams, or about 6 to 9 milligrams of at least one isoflavone per gram of the formulation, wherein the amount of the soy formulation is sufficient to provide greater than 60 milligrams of at least one isoflavone per serving.

The food products of the present invention may comprise various amounts of soy protein. For example, in alternate embodiments, a food product of the present invention may comprise: (i) a soy formulation comprising about 3 to 23 milligrams, or about 5 to 15 milligrams, or about 6 to 9 milligrams of at least one isoflavone per gram of the formulation; and (ii) about 0.4 to 1.2 grams, or about 0.4 to 0.9 grams, or about 0.6 to 0.8 grams protein per gram of the formulation, where the amount of the soy formulation is sufficient to provide greater than 60 milligrams of at least one isoflavone per serving.

In another embodiment, a food product of the present invention may comprise a soy formulation produced by combining a first portion of higher isoflavone concentration soy product produced from the heart of the soy bean, and a second portion of lower isoflavone concentration soy product produced from the whole soy bean. In alternate embodiments, a food product made in this manner may achieve a soy formulation comprising about 3 to 23 milligrams, or about 5 to 15 milligrams, or about 6 to 9 milligrams of at least one isoflavone per gram of the formulation, where the amount of the soy formulation in the food product is sufficient to provide greater than 60 milligrams of at least one isoflavone per serving.

The food products may use soy formulations comprising the isoflavone components and/or their ratios as described herein. Thus, in certain embodiments, the amount of soy formulation utilized in the food product of the present invention may be an amount sufficient to provide well above 60 milligrams of at least one isoflavone per serving. For example, in alternate embodiments of a food product of the present invention, the amount of soy formulation utilized may be an amount sufficient to provide about 120-200 milligrams of at least one isoflavone per serving, or greater than about 200 milligrams of at least one isoflavone per serving, or about 400 to 900 milligrams of at least one isoflavone per serving, or greater than 1800 milligrams of at least one isoflavone per serving, or about 1800 to 2200 milligrams of at least one isoflavone per serving.

Embodiments of the food products of the present invention may comprise a single isoflavone or a plurality of isoflavones. Thus, embodiments of the food products of the present invention may include at least one of diadzin, genistin, or glycitin. In certain embodiments, a plurality of isoflavones including diadzin, genistin, or glycitin may be used. In certain embodiments, a food product of the present invention may comprise a soy formulation comprising diadzin, genistin and glycitin and having a diadzin to genistin to glycitin ratio of between 3:1:2 and 3:4.5:1. In one embodiment, the soy formulation has a ratio of diadzin to genistin to glycitin of approximately 2:1:1, respectively, such that diadzin is the major isoflavone component. In this embodiment, the food product may provide any milligram level of at least one isoflavone per serving, thus differing amounts of the soy formulation may be utilized in the food product.

For example, in alternate embodiments, a food product of the present invention may comprise: about 20 to 40% by weight protein, wherein the protein is provided by a soy formulation of the present invention; about 10 to 80%, by weight carbohydrate; and about 1 to 10%, by weight fat.

A food product of the present invention may further comprise additional components. Thus, in alternate embodiments, a food product of the present invention may comprise at least one of a preservative, a flavoring, a mineral and the like, including but not limited to calcium phosphate, soy lecithin, salt, potassium, chloride, an artificial and/or natural flavor, carragenenan, carboxymethylcellulose, xantham gum, water or milk. Among the carbohydrates suitable for use in the present invention are included fructose, glucose, dextrose, maltodextrin and corn syrup solids.

A food product of the present invention may also be produced in a lower calorie form by substituting an artificial sweetener for all or a portion of the sugars. Suitable artificial sweeteners include sucralose (SPLENDA™), aspartame, saccharin and SinetK™ (acesulfurame K). Plant derived sweeteners such as stevia are also suitable.

A food product of the present invention may take many forms, including a powder for dispersing in a liquid, a tablet, a bar, liquid drinks, a cereal etc. By way of example, a powdered food product of the present invention may comprise: 30 to 32% by weight a soy formulation of the present invention; 55 to 57% by weight carbohydrate; 3 to 5% by weight fat; 0.2 to 1% by weight calcium; 0.2 to 1% by weight phosphorous; 0.1 to 0.7% by weight sodium; 0.2 to 1% by weight potassium; and include ingredients such as fructose, sugar, cocoa, calcium phosphate, maltodextrin, soy lecithin, salt, potassium chloride, artificial flavor, carrageenan, carboxymethyl cellulose and xanthan gum, wherein the food product provides greater than 60 milligrams, or, in certain embodiments, 120-200 milligrams of at least one isoflavone per serving, and having a plurality of isoflavones in the ratios discussed above.

A food product of the present invention may further include vitamins and minerals in an amount of up to 100% or more of the recommended daily allowance for each vitamin. In an embodiment, the food product may comprise biotin. For example, the soy formulation may comprise an amount of biotin such that one dose (or serving) of the food product comprises about 0.5 to 20, or 1 to 10, or about 2.5 mg of biotin.

Alternatively or additionally, the food product may comprise a specific vitamin formulation. For example, a multivitamin may be included. In an embodiment, the food product used for skin treatment may comprise a multi-vitamin or a vitamin supplement. In alternate embodiments, the food product or the mulit-vitamin used in the food product may comprise at least one of a multi-vitamin/multi-mineral supplement, an antioxidant supplement, an amino acid supplement, a skin matrix support supplement, a skin clearing supplement, a fatty acid supplement, or a mood-enhancing supplement. For example, a multi-vitamin or supplements as described herein (Table 1) may be included. In one embodiment, a food product of the present invention may include 20-40% of the recommended daily allowance of most minerals.

Pharmacological Compositions

The present invention may also comprise soy formulations utilized in pharmacologically-active compositions. The pharmacologically-active compositions of the present invention may comprise an amount of the soy formulation so as to be useful for maintaining and/or improving the quality of at least one of epidermal, dermal or subcutaneous tissue, including skin, hair, and nails. In certain embodiments, the pharmacological compositions of the present invention may include a defined range of natural plant estrogens (referred to herein as isoflavones or phytoestrogens) per gram of protein. The pharmacological composition may comprise an amount of isoflavone, protein, and/or other components of the soybean in variety of ranges that may be useful for maintaining and/or improving the quality of at least one of epidermal, dermal or subcutaneous tissue. In an embodiment, the amount of isoflavones and soy protein may be varied by altering the relative amounts of the soybean heart and the rest of the soybean used to prepare the pharmacological composition.

The amount of soy formulation utilized in a pharmacological composition of the present invention may depend on the level of isoflavones desired per dose of the pharmacological composition. In one embodiment, a pharmacological composition of the present invention may include greater than 60 milligrams of at least one isoflavone per serving. In alternate embodiments, the pharmacological composition may comprise greater than about 200 milligrams, or greater than about 1800 milligrams of at least one isoflavone per dose. Thus, for example, a pharmacological composition of the present invention may comprise 9 grams of a soy formulation comprising 7 milligrams of at least one isoflavone per gram of the formulation to provide 63 milligrams of isoflavones. To achieve higher levels of isoflavones, the pharmacological composition may include additional amounts of a soy formulation and/or a soy formulation having a higher level of isoflavones per gram.

Thus, in alternate embodiments, a pharmacological composition of the present invention may comprise a soy formulation comprising about 3 to 23 milligrams, or about 5 to 15 milligrams, or about 6 to 9 milligrams of at least one isoflavone per gram of the formulation, where the amount of the soy formulation is sufficient to provide greater than about 60 milligrams, or greater than about 200 milligrams, or greater than about 1800 milligrams of at least one isoflavone per dose.

The pharmacological compositions of the present invention may comprise various amounts of soy protein. For example, in alternate embodiments, a pharmacological composition of the present invention may comprise: (i) a soy formulation comprising about 3 to 23 milligrams, or about 5 to 15 milligrams, or about 6 to 9 milligrams of at least one isoflavone per gram of the formulation; and (ii) about 0.4 to 1.2 grams, or about 0.4 to 0.9 grams, or about 0.6 to 0.8 grams protein per gram of the formulation, where the amount of the soy formulation is sufficient to provide greater than about 60 milligrams, or greater than about 200 milligrams, or greater than about 1800 milligrams of at least one isoflavone per dose.

In another embodiment, a pharmacological composition of the present invention may comprise a soy formulation produced by combining a first portion of higher isoflavone concentration soy product produced from the heart of the soy bean and a second portion of lower isoflavone concentration soy product produced from the whole soy bean. In alternate embodiments, a pharmacological composition made in this manner may achieve a soy formulation comprising about 3 to 23 milligrams, or about 5 to 15 milligrams, or about 6 to 9 milligrams of at least one isoflavone per gram of the formulation, where the amount of the soy formulation in the pharmacological composition is sufficient to provide greater than about 60 milligrams, or greater than about 200 milligrams, or greater than about 1800 milligrams of at least one isoflavone per dose.

In certain embodiments, the pharmacological compositions of the present invention utilize soy formulations comprising the isoflavone components and/or their ratios as described herein. In certain embodiments, the amount of soy formulation utilized in the pharmacological composition of the present invention may be an amount sufficient to provide well above 60 milligrams of at least one isoflavone per serving. For example, alternate embodiments of a dietary supplement of the present invention may comprise an amount of soy formulation sufficient to provide about 120-200 milligrams of at least one isoflavone per dose, or greater than about 200 milligrams of at least one isoflavone per dose, or about 400 to 900 milligrams of at least one isoflavone per dose, or greater than about 1800 milligrams of at least one isoflavone per dose; or about 1800 to 2200 milligrams of at least one isoflavone per dose.

Embodiments of the pharmacological compositions of the present invention may comprise a single isoflavone or a plurality of isoflavones. Thus, embodiments of the pharmacological compositions of the present invention may include at least one of diadzin, genistin, or glycitin. Or, a plurality of isoflavones including diadzin, genistin, or glycitin may be used. In alternate embodiments, a pharmacological composition of the present invention comprises a soy formulation comprising diadzin, genistin and glycitin and having a diadzin to genistin to glycitin ratio of between 3:1:2 and 3:4.5:1. In an embodiment, the soy formulation may have a ratio of diadzin to genistin to glycitin of approximately 2:1:1, respectively, such that diadzin is the major isoflavone component. In this embodiment, the pharmacological composition may provide any milligram level of at least one isoflavone per dose, thus differing amounts of the soy formulation may be utilized in the pharmacological composition.

A pharmacological composition of the present invention may further include vitamins and minerals in an amount of up to 100% or more of the recommended daily allowance for each vitamin. In an embodiment, the pharmacological composition may comprise biotin. For example, the soy formulation may comprise an amount of biotin such that one dose (or serving) of the pharmacological composition comprises about 0.5 to 20, or 1 to 10, or about 2.5 mg of biotin.

Alternatively or additionally, the pharmacological composition may comprise a specific vitamin formulation. For example, a multivitamin may be included. In an embodiment, the pharmacological composition used for skin treatment may comprise a multi-vitamin or a vitamin supplement. In alternate embodiments, the pharmacological composition or the multi-vitamin used in the pharmacological composition may comprise at least one of a multi-vitamin/multi-mineral supplement, an antioxidant supplement, an amino acid supplement, a skin matrix support supplement, a skin clearing supplement, a fatty acid supplement, or a mood-enhancing supplement. For example, a vitamin or supplements as described herein (Table 1) may be included. In one embodiment, a pharmacological composition of the present invention may include 20-40% of the recommended daily allowance of most minerals.

In certain embodiments, the present invention may comprise a pharmacological composition comprising a soy formulation of the present invention and further comprising a medicinal composition to comprise a pharmacological-medicinal composition. Suitable medicinal compositions include, but are not limited to, the medicinal compositions, drugs and/or prescription drugs used to maintain and/or improve the quality of at least one of epidermal, dermal, or subcutaneous tissue. Or, the composition may comprise medicinal compositions utilized in estrogen replacement therapy; hormone replacement therapy; cholesterol lowering therapy; bone strengthening therapy; endometrial therapy; cancer therapy, including chemotherapy; Alzheimer Disease therapy; ulcer therapy; prostrate therapy; renal therapy; blood therapy; lymphatic therapy; lung therapy; nervous system therapy; diabetes therapy; eye therapy and the like. These medicinal compositions may include PREMARIN®; Fosama; Raloxifene; Tamoxifen; SERM's (selective estrogen receptor modulators) and other drugs known to those of ordinary skill in the art.

The amount of the medicinal composition utilized in this embodiment of a pharmacological composition of the present invention may be an amount sufficient to achieve the desired therapeutic effect while minimizing side or adverse effects. In an embodiment, the amount of the medicinal composition utilized in a pharmacological composition of the present invention will be the same or less than the amount utilized in conventional therapy in the absence of the soy formulation of the present invention.

In certain embodiments, an advantage of a pharmacological composition of the present invention comprising a soy formulation of the present invention and a medicinal composition is that the combination may have synergistic effects. Therefore it may be possible to use a lesser amount of the medicinal composition in a pharmacological composition of the present invention than the amount traditionally utilized in the absence of a soy formulation of the present invention, while achieving substantially the same desired therapeutic effects. This feature also may provide an additional advantage of reducing side or adverse effects caused by the medicinal composition due to the lower amount of the medicinal composition utilized.

Further details, and specific examples of possible uses of pharmacological compositions of the present invention that comprise a soy formulation of the present invention and a medicinal composition are set forth below with reference to the methods of the present invention.

Thus, alternate embodiments of a pharmacological-medicinal composition of the present invention may comprise a soy formulation comprising about 3 to 23 milligrams, or about 5 to 15 milligrams, or about 6 to 9 milligrams of at least one isoflavone per gram of the formulation, where the amount of the soy formulation may be sufficient to provide greater than about 60 milligrams, or greater than about 200 milligrams, or greater than about 1800 milligrams, of at least one isoflavone per dose; and including a medicinal composition wherein the amount of the medicinal composition is sufficient to achieve a desired therapeutic effect.

In other embodiments, a pharmacological-medicinal composition of the present invention may comprise a soy formulation comprising: (i) about 3 to 23 milligrams, or about 5 to 15 milligrams, or about 6 to 9 milligrams of at least one isoflavone per gram of the formulation; and (ii) about 0.4 to 1.2 grams, or about 0.4 to 0.9 grams, or about 0.6 to 0.8 grams protein per gram of the formulation; and (iii) a medicinal composition, wherein the amount of the soy formulation is sufficient to provide greater than about 60 milligrams, or greater than about 200 milligrams, or greater than about 1800 milligrams of at least one isoflavone per dose and wherein the amount of the medicinal composition is sufficient to achieve a specific desired therapeutic effect.

In other embodiments, the pharmacological-medicinal compositions of the present invention may comprise a soy formulation produced by combining a first portion of higher isoflavone concentration soy product produced from the heart of the soy bean and a second portion of lower isoflavone concentration soy product produced from the whole soy bean. When prepared in this manner, the pharmacological-medicinal composition may comprise, in alternate embodiments, about 3 to 23 milligrams, or about 5 to 15 milligrams, or about 6 to 9 milligrams of at least one isoflavone per gram of the formulation wherein the amount of the soy formulation in the pharmacological composition is sufficient to provide greater than about 60 milligrams, or greater than about 200 milligrams, or greater than about 1800 milligrams of at least one isoflavone per dose, and a medicinal composition, wherein the amount of the medicinal composition is sufficient to achieve a desired therapeutic effect.

The isoflavone components and/or their ratios include those discussed above with reference to the soy formulations of the present invention. In particular embodiments of a pharmacological-medicinal composition of the present invention the amount of soy formulation utilized may be an amount sufficient to provide well above 60 milligrams of at least one isoflavone per dose. In particular embodiments of a pharmacological-medicinal composition the amount of soy formulation utilized may be an amount sufficient to provide about 120-200 milligrams of at least one isoflavone per dose; greater than about 200 milligrams of at least one isoflavone per dose; or about 400 to 900 milligrams of at least one isoflavone per dose; or greater than about 1800 milligrams of at least one isoflavone per dose; or about 1800 to 2200 milligrams of at least one isoflavone per dose.

Embodiments of the pharmacological-medicinal compositions of the present invention may comprise a single isoflavone, or a plurality of isoflavones. Thus, embodiments of the pharmacological-medicinal composition may include at least one of diadzin, genistin and glycitin. Or, a plurality of isoflavones including diadzin, genistin and glycitin may be used. In alternate embodiments the pharmacological composition of the present invention may comprise a soy formulation comprising diadzin, genistin and glycitin and having a diadzin to genistin to glycitin ratio of between 3:1:2 and 3:4.5:1; and a medicinal composition wherein the amount of the medicinal composition is sufficient to achieve a desired therapeutic effect. Or, the soy formulation has a ratio of diadzin to genistin to glycitin of approximately 2:1:1, respectively, such that diadzin is the major isoflavone component. In this embodiment, the pharmacological-medicinal composition may provide any milligram level of at least one isoflavone per dose, thus differing amounts of the soy formulation may be utilized in the pharmacological composition.

Embodiments of the pharmacological compositions and/or pharmacological-medicinal compositions of the present invention may further comprise a pharmaceutically acceptable (e.g., biologically inert) carrier composition. The carrier may comprise one or more of the following ingredients: a gel composition; a cellulose composition; a starch; a glycol composition; hydroxypropyl methylcellulose; microcrystalline cellulose; polyethylene glycol; and/or sodium lauryl sulfate. Other ingredients known in the art may also be utilized in the carrier composition.

For example, pharmaceutically acceptable carriers may comprise any of the standard pharmaceutically accepted carriers known in the art. The carrier may comprise a diluent. In one embodiment, the pharmaceutical carrier may be a liquid and the soy formulation may be in the form of a solution. In another embodiment, the pharmaceutically acceptable carrier may be a solid in the form of a powder, a lyophilized powder, or a tablet. Or, the pharmaceutical carrier may be a gel, suppository, or cream. In alternate embodiments, the carrier may comprise a microcapsule, a polymer encapsulated cell, or a virus. The compositions of the present invention may also comprise liposome delivery systems, such as small unilamellar vesicles, large unilamellar vesicles, and multilamellar vesicles. Liposomes may be formed from a variety of phospholipids, such as cholesterol, stearylamine, or phosphatidylcholines. Thus, the term pharmaceutically acceptable carrier encompasses, but is not limited to, any of the standard pharmaceutically accepted carriers, such as water, alcohols, phosphate buffered saline solution, sugars (e.g., sucrose or mannitol), oils or emulsions such as oil/water emulsions or a trigyceride emulsion, various types of wetting agents, tablets, coated tablets and capsules.

Administration of the pharmacological compositions of the present invention may employ various routes. Thus, in alternate embodiments, administration may be oral. Or, administration may be topical, intranasal, or as an aerosol. In other embodiments, administration may be sublingual, or may employ a time-release capsule. In yet another embodiment, administration may be subcutaneous, or transrectal, as by a suppository or the like. There may also be times when administration may be intra-arterial, intramuscular, intravenous, or the like.

For topical use, lotions, creams, ointments, jellies, solutions or suspensions containing the compounds of the invention may be used. Topical applications may also include mouth washes and gargles. Suitable preservatives, antioxidants such as BHA and BHT, dispersants, surfactants, or buffers may be used. Suitable formulations for lotions and creams are described, for example, in U.S. Pat. Nos. 4,760,096, 5,116,605, 5,871,743, each of which are incorporated by reference herein in their entireties.

In certain embodiments, the pharmacological compositions of the present invention may be in the form of a solution in a non-toxic parenterally acceptable solvent or vehicle. Among the acceptable vehicles and solvents that may be employed are water, Ringer's solution, 3-butanediol, isotonic sodium chloride solution, or aqueous buffers, as for example, physiologically acceptable citrate, acetate, glycine, histidine, phosphate, tris or succinate buffers. The solution may contain stabilizers, antimicrobial agents, surfactants and other components as described herein to protect against chemical or biological degradation and aggregate formation.

Dispersible powders and granules suitable for preparation of an aqueous suspension by the addition of water may provide a pharmacological composition of the present invention having the active compound in admixture with a dispersing agent, suspending agent, and one or more preservatives. Suitable preservatives, dispersing agents, and suspending agents are described above. The pharmacological compositions of the present invention may be provided as a sterile lyophilized powder for injection upon reconstitution with a diluent. The diluent can be water for injection, bacteriostatic water for injection, or sterile saline. The lyophilized powder may be produced by freeze drying a solution of the fusion protein to produce the protein in dry form. As is known in the art, a lyophilized compound may have increased stability and a longer shelf life than a liquid solution. The lyophilized powder (cake) many contain a buffer to adjust the pH, as for example physiologically acceptable citrate, acetate, glycine, histidine, phosphate, tris or succinate buffer. The lyophilized powder may also contain lyoprotectants to maintain its physical and chemical stability. The commonly used lyoprotectants are non-reducing sugars and disaccharides such as sucrose, mannitol, or trehalose.

The pharmacological composition may comprise stabilizers to protect against chemical degradation and aggregate formation. Stabilizers may include, but are not limited to antioxidants (BHA, BHT), buffers (citrates, glycine, histidine), or surfactants (polysorbate 80, poloxamers). The pharmacological composition may also contain antimicrobial preservatives, such as benzyl alcohol and parabens. The pharmacological composition may also contain surfactants to reduce aggregation, such as, but not limited to, Polysorbate 80 and poloxomer. The pharmacological composition may also contain additives (e.g., sugars or saline) to adjust the osmotic pressure to be similar to human blood. The pharmacological composition may also contain bulking agents, such as sugars and disaccharides.

The pharmacological compositions may also be in the form of a oleaginous suspension. This suspension may be formulated according to the known methods using suitable dispersing or wetting agents and suspending agents described above. In addition, sterile, fixed oils are conveniently employed as solvent or suspending medium. For this purpose, any bland fixed oil may be employed using synthetic mono- or diglycerides. Also, oily suspensions may be formulated by suspending the active ingredient in a vegetable oil, for example arachis oil, olive oil, sesame oil or coconut oil, or in a mineral oil such as a liquid paraffin. For example, fatty acids such as oleic acid find use in the preparation of injectables. The oily suspensions may contain a thickening agent, for example beeswax, hard paraffin or cetyl alcohol. These compositions may be preserved by the addition of an anti-oxidant such as ascorbic acid.

The pharmacological compositions of the present invention may also be in the form of oil-in-water emulsions or aqueous suspensions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan.

The pharmacological compositions of the present invention may also comprise aqueous suspensions that contain the active compounds in admixture with excipients. Such excipients may include suspending agents, for example sodium carboxymethylcellulose, methylcellulose, hydroxypropylmethylcellulose, sodium alginate, polyvinylpyrrolidone, gum tragacanth and gum acacia; dispersing or wetting agents, such as a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate.

Methods of using Soy Formulations and Products Including Soy Formulations

In addition to the soy formulations, dietary supplements, food products and pharmacological compositions discussed above, the present invention provides methods for maintaining and/or promoting the health and/or cosmetic appearance of an individual. A method of the present invention for maintaining and/or promoting the health and/or cosmetic appearance of an individual may comprise administration of a soy formulation of the present invention. In alternate embodiments, the method may comprise having the individual ingest greater than 60 milligrams, or greater than 120 milligrams, or about 120-200 milligrams, or greater than 200 milligrams of at least one isoflavone per day. Thus, in at least some embodiments, the soy formulations, dietary supplements, food products and pharmacological compositions of the present invention may be utilized in the methods of the present invention to promote and/or maintain the health and/or cosmetic appearance of an individual. In addition, the soy formulations, dietary supplements and food products provide a dietary means for providing beneficial amounts of an isoflavone, or a plurality of isoflavones, and protein to an individual, without requiring the individual ingests unpalatable or difficult to digest amounts of protein.

For example, in one embodiment, the present invention comprises a method of maintaining and/or improving the quality of at least one of skin, hair, or nails in a subject in need thereof comprising administration of a soy formulation to the subject in an amount determined to comprise an amount that is effective to maintain and/or improve the quality of at least one of the epidermal, dermal or subcutaneous tissue in the subject. In certain embodiments, the soy formulation comprises an amount of isoflavone, protein and/or other components of the soybean that are effective to maintain and/or improve the quality of at least one of the epidermal, dermal or subcutaneous tissue in the subject. The method may also comprise the step of evaluating a subject as being someone who requires or desires to maintain and/or improve at least one of his or her epidermal, dermal or subcutaneous tissue including at least one of skin, hair or nails. Also, the method may comprise a determination that a soy formulation may be beneficial to the subject for additional reasons. In an embodiment, the soy formulation may comprise a first portion produced from the heart of a soybean; and a second portion produced from a whole soy bean; wherein the soy formulation is effective to maintain and/or improve the quality of epidermal tissue.

The method may be utilized in any subject requiring or desiring to maintain and/or improve the quality of his or her epidermal, dermal, or subcutaneous tissue. In one embodiment, the subject may comprise a human. In an embodiment, the subject may comprise a post-menopausal female. Or the subject may comprise a non-human mammal as for example, pets that may have a need for treatment of the skin, nails, hair and/or fur. However, other subject may utilize the methods of the present invention to target a variety of symptoms relating to epidermal, dermal, or subcutaneous tissue as described in detail herein.

The soy formulation may be administered over a period of time, and with a frequency as is required to maintain and/or improve the quality of the tissue being targeted. In one embodiment, the soy formulation is administered at least once per day. The soy formulation may be administered for a duration as is required to maintain and/or improve the quality of the tissue being targeted. In alternate embodiments, the soy formulation may be administered to the subject for about two weeks, or for several months or years. It is an advantage of embodiments of the present invention that the soy formulation may be administered for long periods of time with a variety of beneficial health effects.

Also, as described herein administration of the soy formulation may employ a variety of routes. In an embodiment, as for example, where the soy formulation comprises a pharmacological composition, administration may be oral, intravenous, subcutaneous, or topical. Or, for example where the soy formulation is presented as a dietary supplement or food product, administration may be oral.

Thus, in alternate methods of the present invention, an individual may ingest a soy formulation, a dietary supplement, food product and/or pharmacological composition including a soy formulation, comprising about 3 to 23 milligrams of at least one isoflavone per gram of the formulation, or about 5 to 15 milligrams of at least one isoflavone per gram of the formulation, or about 6 to 9 milligrams of at least one isoflavone per gram of the formulation. In alternate embodiments, the soy formulation utilized in a method of the present invention may further comprise about 0.4 to 1.2 grams, or about 0.4 to 0.9 grams, or about 0.6 to 0.8 grams protein per gram of the formulation.

The isoflavone(s) used in the methods of the present invention may comprise at least one of diadzin, genistin or glycitin. In certain embodiments, diadzin, genistin and glycitin may be used in a ratio of between 3:1:2 and 3:4.5:1 of diadzin, genistin and glycitin. In other embodiments, a ratio of diadzin to genistin to glycitin of approximately 2:1:1 is used, such that diadzin is the major isoflavone component.

Embodiments of the soy formulations of the present invention may be used for a variety of skin-related applications. For example, in alternate embodiments, the soy formulations, dietary supplements, food products and compositions of the present invention may be used to reduce scar formation, reduce facial hair growth in females, improve post-laser resurfacing repair of the skin, protect the skin from sun-sensitizing drugs such as ACCUTANE®, anti-depressants, RETIN-A MICRO®, and the like. Additional aspects of skin, hair, and nails that may be treated and/or improved using the methods and/or compositions of the present invention may include at least one of: loss of skin tone; prior photo-aging damage; future photo-aging damage (i.e., the formulation may be used for photoprotection as an “oral sunscreen”); wrinkling or abnormal pigmentation of the skin; thinness, dullness and slow growth of hair; acne; skin cancer; nails that are rough, ridging, flaking, slow growing or splitting; hair loss (men/women); effects of post-laser resurfacing, pulsed light therapy, or any similar method that uses electromagnetic radiation such as light, laser, radio waves, or ultrasound waves to improve epidermal tissues; effects of post-dermabrasion procedures; effects of post-chemical peels; scarring (with reduction being either prophylactic or retroactive); improved wound healing from any source including burns, cuts and plastic surgery; recovery from injection procedures used to cosmetically treat the skin including administration of BOTOX®, Restalane and the like.

In addition to maintaining and/or improving epidermal, dermal and subcutaneous tissue including skin, hair, or nails, the present invention also provides methods for enhancing health which include digesting a soy formulation, dietary supplement, food item, and/or pharmacological compositions of the present invention which include a soy formulation of the present invention. A method of the present invention may be utilized to promote the health of an individual by reducing menopausal like symptoms, including hot flashes or other types of hormonal-based discomfort, as for example as the result of a hysterectomy, breast cancer or a recent pregnancy. A method of the present invention may be utilized to promote the health of an individual by providing a dietary means of achieving the effects achieved by estrogen therapies.

Thus, methods and compositions of the present invention may also be utilized to promote the health of an individual by reducing the following: hot flashes, vaginal itching/dryness, irritability, mood swings, insomnia, night sweats, nervousness, headaches, and painful intercourse; menstrual problems like cramping, bloating, irritability, and weight gain. The methods and compositions may also increase the time between menstrual periods (i.e., fewer periods per year), decrease fatigue by boosting energy levels and mood, maintain healthy breast tissue, endometrial tissue, and other tissues; preserve a strong and healthy skeletal system (bones and joints); and help support a healthy heart, cardiovascular system, and cholesterol levels. The methods and compositions of the present invention may further be utilized to promote the health of an individual by minimizing the effects of Alzheimer's type dementia, age-related loss of cognitive function, and alcoholism. Other potential uses of the methods and compositions of the present invention include: birth control (at higher doses); hormone replacement therapy in combination with mammalian estrogens; breast cancer preventative; prostate cancer preventative; prevention and/or treatment of headaches and migraine headaches; prevention and/or treatment of acne and other skin conditions; improvement of sexual function; lessening effects of chronic fatigue syndrome; and weight loss.

As noted above, and in further detail below, the methods and compositions of the present invention may be utilized to produce health benefits in an individual by alleviating or minimizing symptoms of the following conditions and/or providing health benefits in the following areas:

Skin, Hair and Nails

Aspects of skin, hair, and nails that may be treated and/or improved using the methods, soy formulations, dietary supplements, food products and/or pharmacological compositions of the present invention may include: loss of skin tone; prior photo-aging damage; future photo-aging damage (e.g., photoprotection as an oral sunscreen); wrinkling; abnormal pigmentation; thinness, dullness, or slow growth of hair; facial hair in females; nails that are rough, ridging, flaking, slow growing or splitting; acne; skin cancer; hair loss (men/women); adverse reactions resulting from sensitivity to sun-sensitizing drugs (e.g., ACCUTANE®, anti-depressants, RETIN-A-MICRO®). The methods may also be used to minimize or treat skin damage resulting from post-laser resurfacing, pulsed light therapy or any similar method that uses light, laser, radio waves, or ultrasound waves to treat skin tissue. In other embodiments, the methods of the present invention may also be used to minimize or treat skin damage that may due to dermabrasion procedures, chemical peel, and/or treatment for scar reduction (proactive and retroactive). The methods of the present invention may also be used to improve wound healing due to burns, cuts, plastic surgery or other causes. Also, the methods of the present invention may be used to improve recovery from injection procedures including the administration of BOTOX®, Restatane, and the like.

Thus, in certain embodiments of the methods of the present invention for maintaining and/or improving the quality of at least one of epidermal, dermal or subcutaneous tissue, the method may comprise having an individual ingest a soy formulation of the present invention. In alternate embodiments, the soy formulation is ingested or applied as a dietary supplement, food product, or a pharmacological composition. As described herein, in alternate embodiments, the soy formulation may comprise a soy formulation comprising about 3 to 23 milligrams, or about 5 to 15 milligrams, or about 6 to 9 milligrams of at least one isoflavone per gram of the formulation, where the amount of the soy formulation is sufficient to provide greater than about 60 milligrams, or greater than 200 milligrams, or greater than 1800 milligrams of at least one isoflavone per dose. The pharmacological composition may also include a medicinal composition wherein the amount of the medicinal composition is sufficient to achieve a desired therapeutic effect.

As understood by those of ordinary skill in the art, certain skin or face conditions may result as undesired side effects of a treatment protocol utilizing prescription drugs. Use of a pharmacological composition of the present invention comprising a soy formulation of the present invention and, in certain embodiments, including a medicinal agent such as a prescription drug may advantageously reduce these conditions while also achieving a desired therapeutic effect.

Also, the soy formulation used for skin treatment may include additional compounds. In an embodiment, biotin may be included. For example, the soy formulation may comprise an amount of biotin such that one dose (or serving) of the formulation comprises about 0.5 to 20, or 1 to 10, or about 2.5 mg of biotin.

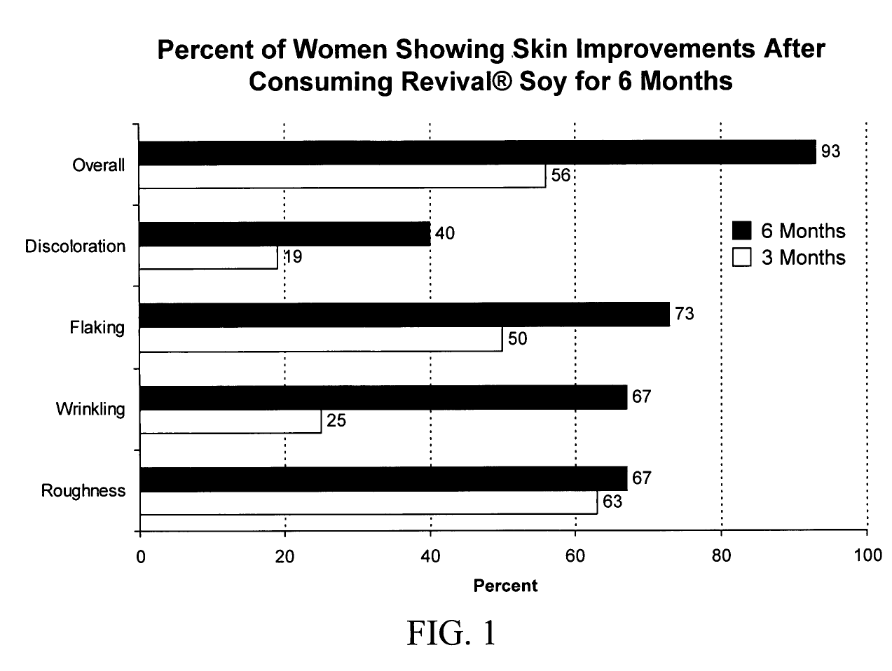

In an embodiment, ingestion of the soy formulation may improve the skin of the subject. For example, ingestion of the soy formulation of the present invention may reduce skin discoloration, flaking, wrinkling, or skin roughness in women consuming the formulation. In alternate embodiments, improvement may be seen after about 3 months or about 6 months (FIG. 1).

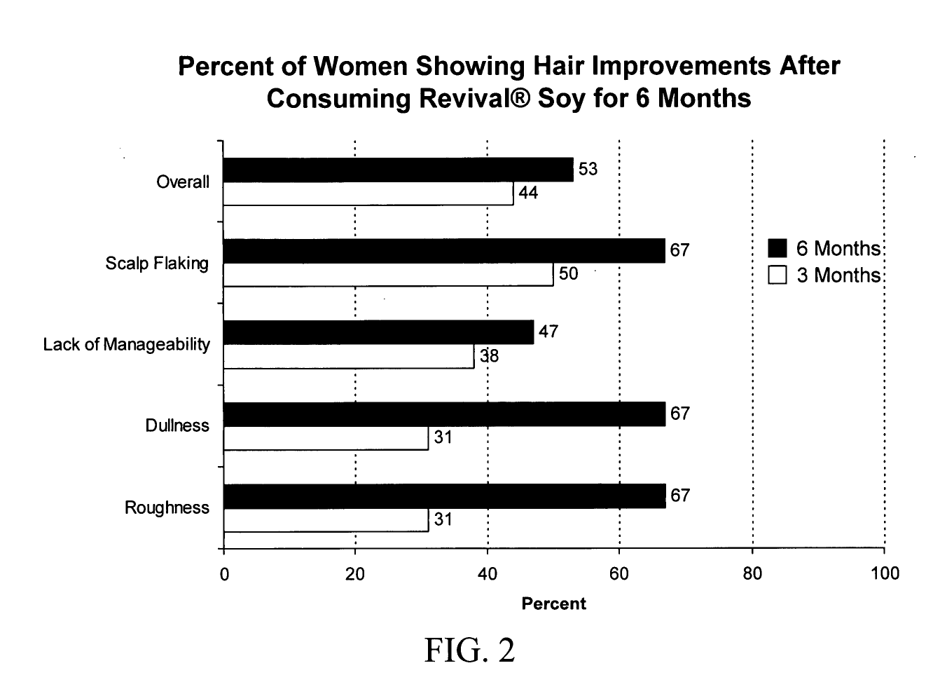

In an embodiment, ingestion of the soy formulation may improve the hair of the subject. For example, ingestion of the soy formulation of the present invention may reduce scalp flaking, improve manageability, reduce dullness, or reduce roughness of the hair in women consuming the formulation. In alternate embodiments, improvement may be seen after about 3 months or about 6 months (FIG. 2).

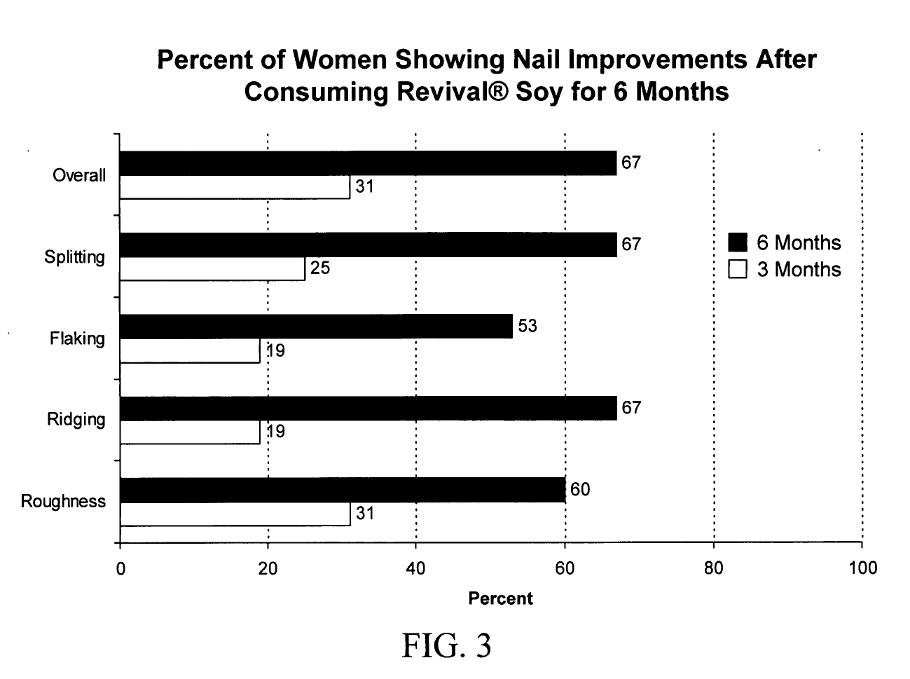

In another embodiment, ingestion of the soy formulation may improve the nails of the subject. For example, ingestion of the soy formulation of the present invention may reduce splitting, flaking, ridging, or roughness of the nails in women consuming the formulation. In alternate embodiments, improvement may be seen after about 3 months or about 6 months (FIG. 3).

In an other embodiment, the composition used for skin treatment may comprise a multi-vitamin or a vitamin supplement. In alternate embodiments, the multi-vitamin or vitamin supplement may comprise at least one of a multi-vitamin/multi-mineral supplement, an antioxidant supplement, an amino acid supplement, a skin matrix support supplement, a skin clearing supplement, a fatty acid supplement, or a mood-enhancing supplement.

The multi-vitamin may comprise multiple vitamins and multiple minerals. Also, the multi-vitamin may comprise at least one of an antioxidant supplement, an amino acid supplement, a skin matrix support supplement, a skin clearing supplement, a fatty acid supplement, or a mood-enhancing supplement. Or, such supplements may be used individually. Examples of such supplements are shown in Table 1. For each of the ingredients it is anticipated that the dose may range from one tenth (i.e., 0.1×) to 10 times (i.e., 10×) of the amounts shown in Table 1. For example, in alternate embodiments, the amount of vitamin C may range from 6 mg to 600 mg (i.e., see Table 1) in the multi-vitamin or vitamin-supplements used in the present invention.

For example, in an embodiment, the soy formulation may comprise a multivitamin or supplement comprising a multi-vitamin/multi-mineral supplement. In alternate embodiments, the multi-vitamin/multi-mineral supplement may include at least one of Vitamin A, Vitamin C, Vitamin D, or Vitamin E (mixed tocopherols & tocotrienols). Additionally or alternatively, the multi-vitamin/multi-mineral supplement may comprise at least one of Thiamin (B1), Riboflavin (B2), Niacin (B3), Vitamin B12, Biotin (B7), Pantothenic Acid (B5), Vitamin B6, or Folate (B9). Additionally or alternatively, the multi-vitamin/multi-mineral supplement may comprise at least one of Choline, Calcium, Iron, Iodine, Zinc, Selenium, Copper, Manganese, Chromium, Molybdenum, Magnesium, or Melatonin.

In an embodiment, the soy formulation may comprise a multi-vitamin or supplement comprising an anti-oxidant supplement. In alternate embodiments, the anti-oxidant supplement may include at least one of Acai Berry, Alpha Lipoic Acid, Astaxanthin, Betatene, Catalase, Cayenne Pepper, Cherry Powder, Citrus Bioflavonoid Complex, Cocoa Extract, Coenzyme Q-10, Curcumin/Tumeric, Flax Lignan Concentrate, Ginger Root, Glutathione Peroxidase, Grape Seed Extract, Green Tea, Hesperidin, Lutein, Lycopene, Maitake Mushroom, Muscadine Grape Seed, Noni Extract, Olive Leaf Extract, Pomegranate, Pycnogenol, Quercetin, Red Wine Extract, Superoxide Dismutase, White Tea, White Willow Bark, Xangold, or Zeaxanthin.

In an embodiment, the soy formulation may comprise a multi-vitamin or supplement comprising an amino acid supplement. In alternate embodiments, the amino acid supplement may include at least one of L-Alanine, L-Arginine, L-Carnitine, L-Cysteine, L-Glutamine, L-Glycine, L-Hydroxyproline, L-Lysine, L-Methionine, L-Proline, L-Serine, L-Tyrosine, or N-Acetyl-Cysteine.

Additionally or alternatively, the soy formulation may comprise a multi-vitamin or supplement comprising a skin matrix support supplement. In alternate embodiments, the skin matrix support supplement may include at least one of Cartilage, Chondroitin Sulfate, Type II Hydrolyzed Collagen, DMAE, Fenugreek, Gelatin, Horse Chestnut, Horsetail Extract, Hyaluronic Acid, Kiwi Seed Extract, Methylsulfonylmethane, MRTin Blend, N-Acetyl-Carnosine, N-Acetyl-Glucosamine, Phosphatidylcholine, Silica, or Silk Peptides.

The soy formulation may also comprise a multi-vitamin or supplement comprising a skin clearing supplement. In alternate embodiments, the skin clearing supplement may include at least one of Broccoli extract, Chrysin, Peppermint Extract, Prickly Pear Cactus Extract, Rosemary, and Yin Zhi Huang.

Additionally or alternatively, the soy formulation may comprise a multi-vitamin or supplement comprising a fatty acid supplement. In alternate embodiments, the fatty acid supplement may include at least one of Borage Oil, Evening Primrose Oil, Fish Oil, and Flax Seed Oil.

Also, the soy formulation may comprise a multi-vitamin or supplement comprising a mood-enhancing supplement. In certain embodiments, the mood-enhancing supplement may comprise St. John's Wort or other known mood enhancing agents.

In an embodiment, the composition may include compounds that help to alleviate menopausal symptoms. PREMARIN® is an example of a medicinal composition that may be used for this embodiment of the method of the present invention The combination of a soy formulation of the present invention and a medicinal composition for hormone replacement therapy and/or estrogen replacement therapy may, in certain embodiments, provide one or more of the following advantages:
1) an additive benefit to skin while reducing menopausal symptoms;
2) improved appearance to hair and/or nails; and/or

3) a reduction in the amount of medicinal composition needed to achieve a therapeutic effect, for example less PREMARIN®, resulting in lower cost to a patient and less risk.

TABLE 1
Vitamin Supplements

| Multi-Vitamin/Multi-Mineral | | | Anti-Oxidants | | |
|---|---|---|---|---|---|
| Item | Dose (100% DV) | | Item | Dose | |
| Vitamin A (Seresis = 2.4 mg b- | 5,000 | IU | Acai Berry | 500 | mg |
| carotene) | | | Alpha Lipoic Acid | 12.5-300 | mg |
| Vitamin C (Seresis = 60 mg) | 60 | mg | Astaxanthin | 2-4 | mg |
| Vitamin D | 400 | IU | Betatene | 50 | mg |
| Vitamin E (tocotrienols) | 30 | IU | Catalase | as required | |
| (Seresis = 6.7 mg) | | | | | |
| Thiamin (B1) | 1.5 | mg | Cayenne Pepper (capsaicin | 85-100 | mg |
| Riboflavin (B2) | 1.7 | mg | for pain) | | |
| Niacin (B3) | 20 | mg | Cherry Powder | 150-1,000 | mg |
| Vitamin B12 | 6 | mcg | Citrus Bioflavonoid Complex | 2.5-1,000 | mg |
| Biotin (B7) | 0.300-2.5 | mg | Cocoa Extract | 300-500 | mg |
| Pantothenic Acid (B5) | 10 | mg | Coenzyme Q-10 | 1-100 | mg |
| Vitamin B6 | 2 | mg | Curcumin/Tumeric | 25-1,440 | mg |
| Folate (B9) | 400 | mcg | Flax Lignan Concentrate | 600 | mg |
| Choline | 50 | mg | (LinumLife) | | |
| Calcium | 1,000 | mg | Ginger Root (GI) | 1.6-150 | mg |
| Iron | 18 | mg | Glutathione Peroxidase | as required | |
| Iodine | 150 | mcg | Grape Seed Extract (Seresis = 50 mg) | 2.5-200 | mg |
| Zinc | 15 | mg | Green Tea | 20-500 | mg |
| Selenium (Seresis = 25 mg) | 70 | mcg | Hesperidin | 2.5-29 | mg |
| Copper | 2 | mg | Lutein (eye) | 1.25-15 | mg |
| Manganese | 2 | mg | Lycopene | 5-20 | mg |
| Chromium | 120 | mcg | Maitake Mushroom (CVD, | 100-250 | mg |
| Molybdenum | 75 | mcg | Cancer) | | |
| Magnesium | 400 | mg | Muscadine Grape Seed | 1,000 | mg |
| Melatonin (evening) | 1-3 | mg | Noni Extract | as required | |
| | | | Olive Leaf Extract | 250-500 | mg |
| | | | Pomegranate (Cancer) | 10-40 | mg |
| | | | Pycnogenol | 0.75-50 | mg |
| | | | Quercetin | 300-750 | mg |
| | | | Red Wine Extract | 10-100 | mg |
| | | | (Resveratrol) | | |
| | | | Superoxide Dismutase | as required | |
| | | | White Tea | 500 | mg |
| | | | White Willow Bark (natural | 200 | mg |
| | | | aspirin) | | |
| | | | Xangold | 50 | mg |
| | | | Zeaxanthin (eye) | 0.66-4 | mg |
| Amino Acid/Peptide | | | | | |
| Supplement | | | Skin Matrix Support | | |
| Item | Dose | | Item | Dose | |
| L-Alanine | 200 | mg | Cartilage (shark, chicken | 200-250 | mg |
| L-Arginine | 25-300 | mg | sternum) | | |
| L-Carnitine (morning) | 75-250 | mg | Chondroitin Sulfate | 1,200 | mg |
| L-Cysteine | 100-500 | mg | Collagen, Hydrolyzed Type II | 1,000 | mg |
| L-Glutamine | 1,500 | mg | DMAE (phosphatidylcholine | 50-130 | mg |
| L-Glycine | 15-250 | mg | synthesis) | | |
| L-Hydroxyproline | 80-1,000 | mg | Fenugreek (FenuLife) | 1,500 | mg |
| L-Lysine | 2.4-500 | mg | Gelatin | 188-1080 | mg |
| L-Methionine | 100-250 | mg | Horse Chestnut (veins) | 250-300 | mg |
| L-Proline | 100-500 | mg | Horsetail Extract (source of | 8.33-880 | mg |
| L-Serine | 10-500 | mg | silica) | | |
| L-Tyrosine | 250 | mg | Hyaluronic Acid | 5-140 | mg |
| N-Acetyl-Cysteine | as required | | Kiwi Seed Extract | 50 | mg |
| | | | Methylsulfonylmethane | 750 | mg |
| | | | (MSM) | | |
| | | | MRTin Blend (shark cartilage, | 250 | mg |
| | | | Ca Phosphate, AstaPro1000) | | |
| | | | N-Acetyl-Carnosine | 500 | mg |
| | | | N-Acetyl-Glucosamine | 25-160 | mg |
| | | | Phosphatidylcholine | 1.5-2,500 | mg |
| | | | Silica | 12.5-90 | mcg |
| | | | Silk Peptides | 50 | mg |
| Skin Clearer | | | Omega Fatty Acids | | |
| Item | Dose | | Item | Dose | |
| Broccoli extract (UGT inducer, | 100 | mg | Borage Oil | 166-2,000 | mg |
| CelluPhase) | | | Evening Primrose Oil | 166-1,000 | mg |
| Chrysin (UGT inducer) | 250-500 | mg | Fish Oil (Virgin Salmon Oil) | 300-3,000 | mg |
| Peppermint Extract | 5-180 | mg | Flax Seed Oil | 25-1,600 | mg |
| Prickly Pear Cactus Extract | 250-600 | mg | | | |
| (HSP inducer) | | | | | |
| Rosemary (HSP inducer) | 2.5-800 | mg | | | |

| | |
|---|---|
| Yin Zhi Huang (Chinese tea, | as required |
| bilirubin clearer) | |

Psychological Boost

| | |
|---|---|
| Item | Dose |
| St. John's Wort | 150-500 mg |

mg = milligram;
mcg = microgram

Menopause/PMS

Menopause symptoms and other hormonal based symptoms that may be treated and/or improved using the methods, formulations, dietary supplements, food products and/or pharmacological compositions of the present invention may include: menopause symptoms such as hot flashes, vaginal itching/dryness, irritability, mood swings, insomnia, night sweats; menopause-like symptoms; post-partum hot flashes; surgically-induced menopause symptoms (i.e., oophorectomy); pre-menstrual (PMS) symptoms; normal menstrual complaints like cramping, bloating, irritability, and weight gain; abnormal vaginal bleeding of any cause; and endometriosis, fibroids or other diseases where blocking estrogen could be beneficial.

In a particular embodiment of a method of the present invention for minimizing or alleviating symptoms of menopause/pms, the method comprises having an individual ingest a pharmacological composition of the present invention. As described herein, in alternate embodiments, the pharmacological composition may comprise a soy formulation comprising about 3 to 23 milligrams, or about 5 to 15 milligrams, or about 6 to 9 milligrams of at least one isoflavone per gram of the formulation, where the amount of the soy formulation is sufficient to provide greater than about 60 milligrams, or greater than 200 milligrams, or greater than 1800 milligrams of at least one isoflavone per dose. The composition may also include a medicinal composition wherein the amount of the medicinal composition is sufficient to achieve a desired therapeutic effect.

For example, a medicinal composition for this embodiment may be a medicinal composition for estrogen replacement therapy or hormone replacement therapy (estrogen+progestins). PREMARIN® is an example of a medicinal composition that may be used for this embodiment of the method of the present invention The combination of a soy formulation of the present invention and a medicinal composition for hormone replacement therapy and/or estrogen replacement therapy may, in certain embodiments, provide one or more of the following advantages:
1) an additive benefit to heart and bone tissues while reducing menopausal symptoms;
2) reduced breast and endometrial proliferation; and/or
3) a reduction in the amount of medicinal composition needed to achieve a therapeutic effect, for example less PREMARIN®, resulting in lower cost to a patient and less risk.

Cardiovascular Related Conditions

Cardiovascular-related conditions that may be treated and/or improved using the methods, formulations, supplements, food products, and/or pharmacological compositions of the present invention may include: lowering total cholesterol, LDL cholesterol, triglycerides and increasing HDL cholesterol or other favorable improvement in lipid profiles; lowering of blood pressure; prevention of myocardial infarction; prevention of second myocardial infarction; prevention or delaying restenosis of coronary bypass grafts or any other vascular grafts; prevention of stroke; and improvement of cardiac stroke volume.

A particular embodiment of a method of the present invention for benefiting heart/cardiovascular related conditions may comprise having an individual ingest a pharmacological composition of the present invention. In alternate embodiments, the pharmacological composition may comprise a soy formulation comprising about 3 to 23 milligrams, or about 5 to 15 milligrams, or about 6 to 9 milligrams of at least one isoflavone per gram of the formulation, where the amount of the soy formulation is sufficient to provide greater than about 60 milligrams, or greater than about 200 milligrams, or greater than about 1800 milligrams of at least one isoflavone per dose. The composition may also include a medicinal composition wherein the amount of the medicinal composition is sufficient to achieve a desired therapeutic effect.

For example, a medicinal composition for this embodiment may comprise LIPITOR® or another cholesterol lowering medication. The combination of a soy formulation of the present invention and a medicinal composition for cholesterol lowering may, in certain embodiments, provide one or more of the following advantages:
1) an additive benefit to heart and bone tissues while generating a better lipid profile; and/or
2) a reduction in the amount of medicinal composition needed to achieve a therapeutic effect, for example less LIPITOR®, resulting in lower cost to a patient and less risk of liver abnormalities caused by LIPITOR®.

Bone/Skeletal System Conditions

Bone and/or skeletal conditions that may be treated and/or improved using the methods, formulations, dietary supplements, food products, and/or pharmacological compositions of the present invention may include: osteoporosis; hip fracture; quicker recovery after hip fracture surgery; disorders of the joints or cartilage; and stimulation of growth and reduction of inflammation of connective tissue/joints.

For example, in one particular embodiment of a method of the present invention for minimizing or alleviating symptoms of bone and/or skeletal conditions, the method comprises having an individual ingest a pharmacological composition of the present invention wherein the pharmacological composition comprises may comprise a soy formulation comprising about 3 to 23 milligrams, or about 5 to 15 milligrams, or about 6 to 9 milligrams of at least one isoflavone per gram of the formulation, where the amount of the soy formulation is sufficient to provide greater than about 60 milligrams, or greater than 200 milligrams, or greater than 1800 milligrams of at least one isoflavone per dose. The composition may also include a medicinal composition wherein the amount of the medicinal composition is sufficient to achieve a desired therapeutic effect.

A medicinal composition for this embodiment may comprise a medicinal composition for strengthening bones, or a medicinal composition for estrogen replacement therapy or hormone replacement therapy (estrogen+progestins). Fosamax, Raloxifene and PREMARIN® are examples of medicinal compositions for this embodiment of the method of the present invention The combination of a soy formulation of the present invention and a medicinal composition may provide one or more of the following advantages:
1) an additive benefit to heart and bone tissues while reducing menopausal symptoms;
2) reduced breast and endometrial proliferation; and/or
3) a reduction in the amount of medicinal composition needed to achieve a therapeutic effect, for example less PREMARIN®, resulting in lower cost to a patient and less risk.

Breast/Prostrate Related Conditions

Breast and/or prostate-related conditions that may be improved and/or treated using the methods, formulations, dietary supplements, food products, and/or pharmacological compositions of the present invention may include: prevention and treatment of any abnormal breast tissue including, but not limited to, fibrocystic disease, ductal hyperplasia, ductal carcinoma in situ (DCIS), locally confined breast cancer or metastatic breast cancer; hot flashes and other menopause-like symptoms caused by breast cancer treatment or preventative treatment (e.g. hot flashes caused by use of tamoxifen); quality and duration of life after diagnosis of breast tumor; early childhood treatment with isoflavones for prevention of breast cancer/prostate cancer later in life; reducing the growth rate of cancerous tissue/cells; and extension of life span after breast/prostate cancer.

In a particular embodiment of a method of the present invention for minimizing or alleviating symptoms of breast/prostrate related conditions, the method comprises having an individual ingest a pharmacological composition of the present invention wherein the pharmacological composition comprises a soy formulation comprising about 3 to 23 milligrams, or about 5 to 15 milligrams, or about 6 to 9 milligrams of at least one isoflavone per gram of the formulation, where the amount of the soy formulation is sufficient to provide greater than about 60 milligrams, or greater than 200 milligrams, or greater than 1800 milligrams of at least one isoflavone per dose. The composition may also include a medicinal composition wherein the amount of the medicinal composition is sufficient to achieve a desired therapeutic effect.

In an embodiment, a medicinal composition for this embodiment is a medicinal composition for estrogen replacement therapy or hormone replacement therapy (estrogen+progestins). PREMARIN® is an example of a medicinal composition for this embodiment of the method of the present invention Other medicinal compositions include tamoxifen, raloxifene or SERM's. The combination of a soy formulation of the present invention and a medicinal composition may provide one or more of the following advantages:
1) an additive benefit to heart and bone tissues while having a preventive effect for breast cancer;
2) reduced breast and endometrial proliferation; and/or

3) a reduction in the amount of medicinal composition needed to achieve a therapeutic effect, for example less PREMARIN®, resulting in lower cost to a patient and less risk.

Endometrium

Endometrium-related conditions that may be improved and/or treated using the methods, formulations, dietary supplements, food products, and/or pharmacological compositions of the present invention may include: endometrial abnormalities or disease; and endometrial hyperplasia/cancer caused by medications that stimulate the endometrium (e.g. tamoxifen).

In a particular embodiment of a method of the present invention for preventing and/or minimizing endometrial conditions, the method comprises having an individual ingest a pharmacological composition of the present invention wherein the pharmacological composition comprises a soy formulation comprising about 3 to 23 milligrams, or about 5 to 15 milligrams, or about 6 to 9 milligrams of at least one isoflavone per gram of the formulation, where the amount of the soy formulation is sufficient to provide greater than about 60 milligrams, or greater than 200 milligrams, or greater than 1800 milligrams of at least one isoflavone per dose. The composition may also include a medicinal composition wherein the amount of the medicinal composition is sufficient to achieve a desired therapeutic effect.

In alternate embodiments, a medicinal composition for this embodiment is a medicinal composition comprising tamoxifen, raloxifene and/or SERM's. The combination of a soy formulation of the present invention and a medicinal composition may provide one or more of the following advantages:
1) an additive benefit to heart and bone tissues while treating endometrial conditions;
2) reduced breast and endometrial proliferation, and prevention of endometrial hyperplasia or cancer (tamoxifen has been shown to promote formation of endometrial cancers, the soy formulation of the present invention may reduce this risk); and/or
3) a reduction in the amount of medicinal composition needed to achieve a therapeutic effect, for example less PREMARIN®, resulting in lower cost to a patient and less risk.

Head/Brain Symptoms

Conditions related to the head and/or brain that may be improved and/or treated using the methods, formulations, dietary supplements, food products, and/or pharmacological compositions of the present invention may include: prevention and treatment of Alzheimer's or other diseases of cognition; macular degeneration; migraine/vascular-related headaches; anxiety, nervousness, depression or other similar affective disorders; hereditary hemorrhagic talengiectasia (HHT); male pattern baldness and female baldness; and improvement in cognitive function.

In a particular embodiment of a method of the present invention for minimizing or alleviating head/brain symptoms, the method may comprise having an individual ingest a pharmacological composition of the present invention wherein the pharmacological composition comprises a soy formulation comprising about 3 to 23 milligrams, or about 5 to 15 milligrams, or about 6 to 9 milligrams of at least one isoflavone per gram of the formulation, where the amount of the soy formulation is sufficient to provide greater than about 60 milligrams, or greater than 200 milligrams, or greater than 1800 milligrams of at least one isoflavone per dose. The composition may also include a medicinal composition wherein the amount of the medicinal composition is sufficient to achieve a desired therapeutic effect.

In an embodiment, a medicinal composition for this embodiment is a medicinal composition for Alzheimer's disease. The combination of a soy formulation of the present invention and a medicinal composition may provide one or more of the following advantages:
1) an additive benefit to heart and bone tissues while treating Alzheimer's symptoms;
2) a reduction in the amount of medicinal composition needed to achieve a therapeutic effect, resulting in lower cost to a patient and less risk.

Gastrointestinal (GI) Tract Conditions

Gastrointestinal (GI) tract conditions that may be improved and/or treated using the methods, formulations, dietary supplements, food products, and/or pharmacological compositions of the present invention may include: constipation; peptic ulcers or other ulcers; gastroesophageal reflux (GERD); any inflammatory bowel diseases such as ulcerative colitis or Crohn's disease; and cancers of the GI tract such as colon cancer.

Other conditions that may be treated using the methods, soy formulations, dietary supplements, food products, and pharmacological compositions of the present invention include the following. In an embodiment, the pharmacological composition may include a medicinal composition targeted towards the particular condition.

Kidney Related Conditions

Prevention of disease, particularly diabetic nephropathies, polycystic kidney disease; improvement in kidney function such as increasing glomerular filtration rate (GFR); or management of lipid abnormalities secondary to renal disease.

Lung and Breathing Related Conditions

Improvement in elasticity or treatment of cancers.

Additional Prostate/Urinary Tract Conditions

Any prostate disorders; prevention/treatment of bladder or other reproductive tract cancers in men and women; prevention of death from prostate cancer (Japanese men have equal incidence of prostate cancer, but death rate is very low); treatment/prevention of symptoms of benign prostatic hyperplasia (BPH) (e.g., urgency, frequency, painful ejaculation, nocturia) and prostate cancer; prevention of prostrate cancer progression; treatment for impotence; lowering prostate specific antigen (PSA); lowering circulating dihydrotestosterone (DHT) levels; or inhibition of 5-alpha reductase.

Immune System Conditions

Autoimmune/rheumatological disorders such as sarcoidosis, rheumatoid arthritis, lupus; or boosting immune system of immunocompromised individuals.

Reproductive System Conditions

Increasing length of time between menstrual cycles.; birth control at higher doses; or increased fertility by causing regular menstrual cycles.

Nervous System Conditions

Treatment of pain, minimizing pain associated with trauma/surgery; or treatment/minimization of nerve damage associated with trauma/surgery.

Thus, it has been found that having mice ingest a soy formulation shortens time of neuropathies induced by nerve damage. Thus, for example, a breast cancer patient could take soy before surgery to minimize nerve damage/potential pain from surgery.

As understood by those of ordinary skill in the art, nervous system conditions/pain are often treated utilizing prescription drugs. Use of a pharmacological composition of the present invention comprising a soy formulation of the present invention and the prescription drug may advantageously provide the foregoing advantages thus reducing the amount of prescription drug necessary to achieve the desired therapeutic effect.

Diabetes Associated Diseases

Prevention of diabetic retinopathies; prevention/treatment of heart disease; prevention/treatment of nerve damage; or prevention/treatment of kidney disease.

Eye Conditions

Prevention of cataracts and macular degeneration.

General Conditions

Improvement in sexual function—men and women; obesity; treatment for chronic fatigue syndrome and fibromyalgia; or treatment of hypo/hyperglycemia.

For example, in a particular embodiment of a method of the present invention for achieving these health benefits, the method may comprise having an individual ingest a pharmacological composition of the present invention wherein the pharmacological composition comprises a soy formulation comprising about 3 to 23 milligrams, or about 5 to 15 milligrams, or about 6 to 9 milligrams of at least one isoflavone per gram of the formulation, where the amount of the soy formulation is sufficient to provide greater than about 60 milligrams, or greater than 200 milligrams, or greater than 1800 milligrams of at least one isoflavone per dose. The composition may also include a medicinal composition wherein the amount of the medicinal composition is sufficient to achieve a desired therapeutic effect.

In an example embodiment, a medicinal composition for treatment of prostate/urinary tract conditions may be a medicinal composition for estrogen replacement therapy or hormone replacement therapy (estrogen+progestins). PREMARIN® is an example of a medicinal composition for this embodiment of the method of the present invention. The combination of a soy formulation of the present invention and a medicinal composition for hormone replacement therapy and/or estrogen replacement therapy may provide one or more of the following advantages:
1) an additive benefit to heart and bone tissues while reducing menopausal symptoms;
2) reduction of estrogenic side effects (enlarged breasts, decreased libido, feminization);
3) reduced breast and endometrial proliferation; or
4) a reduction in the amount of medicinal composition needed to achieve a therapeutic effect, for example less PREMARIN®, resulting in lower cost to a patient and less risk.

The following examples illustrate the production of a soy formulation of the present invention which may be utilized in a dietary supplement and/or food product of the present invention and/or in a method of the present invention.

### EXAMPLES

### Example 1

### Soy Formulations

The following is one example of a blend of a first portion of higher isoflavone concentration soy product from the heart of the bean and a second portion of lower isoflavone concentration soy product to achieve a natural soy formulation of the present invention having an milligram isoflavone to gram soy protein ratio and a diadzin to genistin to glycitin ratio falling in the certain of the ranges of the formulation of the present invention.

Soy product derived from the heart of the soybean produces relatively higher concentrations of isoflavones (24-36 mg of isoflavones per gram of protein). The higher isoflavone concentration soy product produced from the heart of the soy bean used in the present invention contains Diadzin/Diadzein:Genistin/Genistein:Glycitin/Glycitein in the average ratios of 3.33:1.00:2.33, respectively (Table 2). Soy derived from the whole soybean, such as found in soy product produced from the whole soy beans, yields relatively lower concentrations of isoflavones (<1.0-5 mg isoflavones per gram of protein). The lower isoflavone concentration soy product used in the present invention contains Diadzin/Diadzein:Genistin/Genistein:Glycitin/Glycitein in the average ratios of 5.00:10.00:1.00, respectively (Table 2). One example of the invention is a blend of approximately 4 grams of the higher isoflavone concentration soy product and approximately 18.4 grams of the lower isoflavone concentration soy product resulting in Diadzin/Diadzein:Genistin/Genistein:Glycitin/Glycitein in the approximate ratios of 2.00:1.00:1.00, respectively (Table 2).

TABLE 2
AVERAGE RATIOS OF ISOFLAVONES

| | Higher isoflavone | Lower isoflavone | |
|---|---|---|---|
| | concentration | concentration | Blend ratios |
| | soy product | soy product | (approximate) |
| Isoflavone | (4 gm) | (18.4 gm) | (22.4 gm) |
| Diadzin/Diadzein | 3.33 | 5.00 | 2.00 |
| Genistin/ | 1.00 | 10.00 | 1.00 |
| Genistein | | | |
| Glycitin/Glycitein | 2.33 | 1.00 | 1.00 |

Long soybean crop histories and multiple testings of isoflavone concentrations in soy product derived therefrom consistently demonstrate these average ratios of constituent isoflavone concentrations in naturally grown soybeans. Naturally grown soybeans are defined as those without genetic modifications for isoflavone content. Actual ratios of constituent isoflavones in the final blended formulation depend on the relative amounts of higher isoflavone concentration soy product and lower isoflavone concentration soy product combined. The final soy formulation of the present invention may, in certain embodiments, have an isoflavone concentration falling within the ranges discussed above. An example of a final soy composition of the present invention is illustrated in Table 3.

TABLE 3

| | grams | mg isoflavones | |
|---|---|---|---|
| | protein per | per | |
| Isoflavone | gram | gram | Total mg |
| Concentration | formulation | formulation | isoflavones |
| Higher | 4 | 24-36 | 96-144 |
| Lower | 18.4 | <1.0-3.4 | <18.4-62.56 |
| Total | 22.4 | 6-9 | <114.4-206.56 |

### Example 2

### The Effect of Soy on the Health and Appearance of the Skin, Hair, and Nails in Post-Menopausal Women

As described herein, soy and/or soy isoflavones may be beneficial for a variety of dermatologic problems including a reduction in the following: incidence of hair loss; transdermal water loss; fine wrinkles and UV radiation-induced thickening of the epidermis; and skin cancer. Also, soy isoflavones may stimulate collagen production, and topical application of soy isoflavones may reduce sunburn severity and inflammatory edema reactions, and suppress contact hypersensitivity.

There have been no studies examining the effects of dietary soy on dermatologic conditions in post-menopausal women, a population with an increasing interest in the use of soy as an alternative to hormone therapy. Thus, a case study was conducted in which a dietary supplement of the present invention, REVIVAL® Soy, was administered to post-menopausal subjects and the appearance of the skin, hair and nails monitored after 3 months and 6 months. The results are summarized as follows:

SKIN: Significant reductions in skin flaking and discoloration were seen after 3 months, while improvements (i.e., reduction) in both skin wrinkling and discoloration were seen after 6 months of soy consumption.

NAILS: Significance improvements were seen in nail quality with reductions in roughness, ridging, flaking, and splitting, after consuming soy for 6 months.

HAIR: Significant improvements in hair quality, with reductions in roughness and improved manageability seen after 3 months, while improvements in hair roughness, dullness, and overall assessment were seen after 6 months of soy consumption.

Study Design

The study was a single-center study, conducted at a dermatology clinic, was comprised of 40 post-menopausal women 50-65 years of age with mild to moderate photoaging who were not currently consuming soy supplements. The women either consumed one REVIVAL® Soy shake (20 grams soy protein with 160 mg total isoflavones; n=20) per day, or received no dietary intervention (Control; n=20) for 6 months. The subjects were asked to come to the clinic for a total of three visits, at baseline, 3 months and 6 months. During each visit, the subjects were asked to complete a questionnaire in order to provide self-reported improvements. Following completion of the questionnaire, the staff of the clinic examined the subjects to assess the health and appearance of their skin, hair and nails.

To assess the health and appearance of the skin four measurements were conducted. Transepidermal water loss was measured with an evaporimeter, skin hydration was tested with a corneometer, and facial skin pigmentation and fine wrinkles were photographically recorded for later assessment.

To determine the potential beneficial effects of REVIVAL® Soy on hair health and appearance, hair was visually assessed for sheen and tested for ease of combing. To compare treatment differences and improvements over time, the shampoo used by the subjects was standardized. The women were provided a list of acceptable shampoos for use during the study.

To measure changes in the health and appearance of fingernails, the nails were visually appraised for shine, splitting, and flexibility.

To confirm that the study volunteers remained on the same nutritional diets throughout the study, a nutritional assessment was conducted at the beginning, after three months on the study, and at the end of the study (6 months).

The statistical evaluation of the data was performed by Dermatology Consulting Services utilizing a Mann Whitney nonparametric two-tailed paired test. Statistical significance was defined as p less than or equal to 0.05.

Results

The results for the soy supplement study revealed a number of physical benefits from use of the product for the 6-month study period. The study evaluated appearance changes in the face, fingernails, and scalp hair comparing those subjects consuming the soy versus those subjects on their normal self-selected diet devoid of soy products and estrogen supplements. The investigator noted improvement in specific characteristics in each area.

As shown in FIG. 1, the results of this study revealed a number of investigator-assessed physical benefits from use of the product for the 6 month study period. After 3 months of soy supplementation, statistically significant improvements were noted in facial skin flaking, discoloration, and overall appearance. After 6 months of dietary soy consumption, improvements were seen in facial wrinkling, discoloration, and overall appearance. Thus, by the end of month 3, statistically significant improvement was noted in facial skin flaking (p=0.028), discoloration (p=0.045), and overall appearance (p=0.05). At the end of month 6, improvement was seen in the soy group in terms of facial wrinkling (p=0.004), discoloration (p=0.016), and overall appearance (p=0.0001).

FIG. 2 shows results as assessed for the appearance of the subjects' hair. Improvement in hair appearance was also seen by the investigator at both the 3- and 6-month evaluation time points. Improvements were evident, with reductions in hair roughness, and improved manageability and overall appearance after consuming soy for 3 months. Additional improvements were observed after 6 months, with reduced hair roughness, dullness, and improved overall appearance. Thus, there was improvement seen as a decrease in hair roughness (p=0.041), increased manageability (p=0.018), and improved overall appearance (p=0.016) at the end of 3 months of the soy supplement use. Additional improvement was seen at the end of month 6 in terms of reduced hair roughness (p=0.004), reduced dullness (p=0.048), and improved overall appearance (p=0.005).

Also, as shown in FIG. 3, the nails of the subjects using the soy supplement also demonstrated investigator perceived improvement. Improvement was noted in nail roughness, ridging, flaking, splitting, and overall appearance after 6 months of dietary soy; thus, by month 6 improvement was noted as a reduction in nail roughness (p=0.017), a ridging (p=0.006), flaking (p=0.049), splitting (p=0.007), and an improvement in overall appearance (p=0.008).

Similar to investigator-assessed benefits, the study subjects also perceived beneficial changes. Improvement in facial skin was also perceived by the subjects using the soy supplement in terms of reduced facial roughness (p=0.013) and wrinkling (p=0.014) at the end of month 3. There was a continued perception of reduced facial roughness (p=0.009) at the end of month 6 in the soy supplement group. There was also an improvement noted in hair roughness (p=0.037) after 6 months of soy consumption.

Primary Efficacy Endpoint

A statistically significant (p=0.05 or less) improvement in skin flaking and discoloration was seen at the end of month 3 and a statistically significant improvement in skin wrinkling and discoloration was seen at the end of month 6 as assessed by the dermatologist investigator.

A statistically significance improvement was seen in nail roughness, ridging, flaking, splitting, and overall appearance at the end of month 6 as assessed by the dermatologist investigator.

A statistically significant improvement in hair roughness, manageability, and overall assessment was seen at the end of month 3 and a statistically significant improvement in hair roughness, dullness, and overall assessment was seen at the end of month 6 by the dermatologist investigator.

Secondary Efficacy Endpoint

A statistically significant (p=0.05 or less) improvement was seen by the dermatologist investigator at the end of months 3 and 6 in overall facial appearance.

In summary, the results of the study demonstrate that daily soy consumption may improve dermatologic health. Improvements in skin, hair, and nails were evident after 6 months of soy consumption, with some benefits being noted as early as 3 months. These findings point to the value of a soy supplement for the appearance of skin, hair, and nails in postmenopausal women.

Although the invention has been described with reference to particular embodiments and features, other similar embodiments and features may be utilized to obtain similar results. Variations and modifications of the soy formulations, dietary supplements, food products, pharmacological compositions, and methods of the present invention will be apparent to one skilled in the art and the present disclosure is intended to cover all such modifications and equivalents within the scope of the following claims.

What is claimed is:

1. A soy formulation comprising: a first portion produced from the heart of a soybean; and a second portion produced from a whole soy bean; wherein the soy formulation is effective to maintain and/or improve the quality of at least one of epidermal, dermal or subcutaneous tissue.

2. The soy formulation of claim 1, wherein the amount of isoflavone comprises about 3 to 23 milligrams of an isoflavone per gram of the formulation.

3. The soy formulation of claim 1, wherein the isoflavaone comprises at least one of diadzin, genistin or glycitin.

4. The soy formulation of claim 1, wherein the isoflavone comprises diadzin, genistin and glycitin, and wherein the diadzin to genistin to glycitin ratio is between 3:1:2 and 3:4.5:1.

5. The soy formulation of claim 1, wherein the soy formulation further comprises about 0.4 to 1.2 grams protein per gram of the formulation.

6. The soy formulation of claim 1, further comprising biotin.

7. The soy formulation of claim 1, wherein the biotin is present in an amount to provide a single dose ranging from about 1 to 10 mg.

8. The soy formulation of claim 1, further comprising a multi-vitamin or a vitamin supplement.

9. A dietary supplement comprising: about 40 to 90% by weight of protein; about 1 to 59% by weight carbohydrate; and about 1 to 10% by weight fat; wherein the protein comprises the soy formulation of claim 1, and wherein the amount of the soy formulation in the dietary supplement is sufficient to provide greater than 60 milligrams of at least one isoflavone per serving.

10. A food product comprising: 20 to 40% by weight protein; 10 to 80% by weight carbohydrate; and 1 to 10% by weight fat; wherein the protein comprises the soy formulation of claim 1 wherein the amount of the soy formulation in the food product is sufficient to provide greater than 60 milligrams of at least one isoflavone per serving.

11. A pharmacological composition comprising a pharmaceutically acceptable carrier and the soy formulation of claim 1, wherein the amount of the soy formulation in the pharmacological composition is sufficient to provide greater than 60 milligrams of at least one isoflavone per dose.

12. The pharmacological composition of claim 10, further comprising a medicinal composition.

13. A method of maintaining and/or improving the quality of at least one skin, hair or nails in a subject in need thereof comprising:
evaluating a subject as being someone who requires or desires to maintain and/or improve his or her skin, hair or nails; and
applying a soy formulation to the subject in an amount that is effective to maintain and/or improve the quality of at least one of the epidermal, dermal or subcutaneous tissue in the subject.

14. The method, of claim 13, wherein the soy formulation comprises a first portion produced from the heart of a soybean and a second portion produced from a whole soy bean;

15. The method of claim 13, wherein the subject is a post-menopausal human.

16. The method of claim 13, wherein the soy formulation is administered at least once per day.

17. The method of claim 13, wherein administration is topical.

18. The method of claim 13, wherein the administration is oral.

19. The method of claim 13, wherein the amount of isoflavone comprises about 3 to 23 milligrams of an isoflavone per gram of the formulation.

20. The method of claim 13, wherein the isoflavaone comprises at least one of diadzin, genistin or glycitin.

21. The method of claim 13, wherein the isoflavone comprises diadzin, genistin and glycitin, and wherein the diadzin to genistin to glycitin ratio is between 3:1:2 and 3:4.5:1.

22. The method of claim 13, wherein the soy formulation further comprises about 0.4 to 1.2 grams protein per gram of the formulation.

23. The method of claim 13, wherein the soy formulation further comprises biotin.

24. The method of claim 13, wherein the soy formulation further comprises a multi-vitamin or a vitamin supplement.

25. The method of claim 13, wherein the formulation is applied to reduce at least one of loss of skin tone, photo-aging of the skin, wrinkling of the skin, acne, skin cancer, adverse reactions of the skin resulting from sensitivity to sun-sensitizing drugs, facial hair in females, or abnormal pigmentation of the skin.

26. The method of claim 13, wherein the formulation is applied to reduce at least one of thinness of hair; dullness of hair; slow growth of hair, or hair loss.

27. The method of claim 13, wherein the formulation is applied to reduce at least one of roughness of at least one nail, ridging of at least one nail, flaking of at least one nail, slow growing of at least one nail, or splitting of at least one nail.

28. The method of claim 13, wherein the formulation is applied to reduce the effect at least one of exposure of the skin to light, laser, radiowaves, or ultrasound waves.

29. The method of claim 13, wherein the formulation is applied to reduce skin damage occurring upon at least one of a dermabrasion procedure, a chemical peel procedure, treatment for scar reduction, or injection procedures used to cosmetically treat the skin.

30. The method of claim 13, wherein the formulation is applied to reduce to improve wound healing.
